(19) United States
(12) Patent Application Publication
(10) Pub. No.: US 2009/0221539 A1
(43) Pub. Date: Sep. 3, 2009

(54) METHOD OF DETECTING AND REDUCING BOAR TAINT USING NUCLEAR RECEPTORS

(75) Inventors: E. James Squires, Guelph (CA); John Peacock, Thornhill (CA); James Gilmore, Guelph (CA); Philip Sinclair, Ottawa (CA); Michael Terner, Richmond Hill (CA)

Correspondence Address: MCKEE, VOORHEES & SEASE, P.L.C., 801 GRAND AVENUE, SUITE 3200, DES MOINES, IA, 50309-2721, US

(73) Assignee: UNIVERSITY OF GUELPH, GUELPH, ONTARIO (CA)

(21) Appl. No.: 12/097,293

(22) PCT Filed: Dec. 13, 2006
(86) PCT No.: PCT/CA06/02040

§ 371 Date: Oct. 27, 2008

Related U.S. Application Data
(60) Provisional application No. 60/750,595, filed on Dec. 15, 2005.

(51) Int. Cl. A61K 31/56; C12Q 1/68; A61K 31/497; A61K 31/415; C07H 21/04

(52) U.S. Cl. 514/171; 435/6; 514/254.11; 514/391; 536/23.1

(57) ABSTRACT

A method for preventing or reducing boar taint, a method of screening pigs to determine those more likely to have reduced boar taint and a method for screening substances that enhance skatole metabolism or androstenone in a pig, are disclosed These methods involve the use of the nuclear receptors constitutive androstane receptor, pregnane X receptor and farnesoid X receptor, which have been found to modulate the activity of the enzymes involved in skatole and/or androsterone metabolism

### FIELD OF THE INVENTION

The invention relates to methods for detecting, determining susceptibility to and preventing boar taint.

### BACKGROUND OF THE INVENTION

Male pigs that are raised for meat production are usually castrated shortly after birth to prevent the development of off-odors and off flavors (boar taint) in the carcass. Boar taint is primarily due to high levels of either the 16-androstene steroids (especially androstenone) or skatole in the fat. Recent results of the EU research program AIR 3-PL94-2482 suggest that skatole contributes more to boar taint than androstenone (Bonneau, M., 1997).

Skatole is produced by bacteria in the hindgut which degrade tryptophan that is available from undigested feed or from the turnover of cells lining the gut of the pig (Jensen and Jensen, 1995). Skatole is absorbed from the gut and metabolized primarily in the liver (Jensen and Jensen, 1995). High levels of skatole can accumulate in the fat, particularly in male pig, and the presence of a recessive gene Ska¹, which results in decreased metabolism and clearance of skatole has been proposed (Lundstrom et al., 1994; Friis, 1995). Skatole metabolism has been studied extensively in ruminants (Smith, et al., 1993), where it can be produced in large amounts by ruminal bacteria and results in toxic effects on the lungs (reviewed in Yost, 1989). Environmental and dietary factors affecting skatole levels (Kjeldsen, 1993; Hansen et al., 1995) but do not sufficiently explain the reasons for the variation in fat skatole concentrations in pigs. Claus et al. (1994) proposed high fat skatole concentrations are a result of an increased intestinal skatole production due to the action of androgens and glucocorticoids. Lundström et al. (1994) reported a genetic influence on the concentrations of skatole in the fat, which may be due to the genetic control of the enzymatic clearance of skatole. The liver is the primary site of metabolism of skatole and liver enzymatic activities could be the controlling factor of skatole deposition in the fat. Baek et al. (1995) described several liver metabolites of skatole deposition in the fat. Baek et al. (1995) described several liver metabolites of skatole found in blood and urine with the major being MII and MIII. MII, which is a sulfate conjugate of 6-hydroxyskatole (pro-MII), was only found in high concentrations in plasma of pigs which were able to rapidly clear skatole from the body, whereas high MIII concentrations were related to slow clearance of skatole. Thus the capability of synthesis of MII could be a major step in a rapid metabolic clearance of skatole resulting in low concentrations of skatole in fat and consequently low levels of boar taint.

In view of the foregoing, further work is needed to fully understand the metabolism of skatole in pig liver and to identify the key enzymes involved. Understanding the biochemical events involved in skatole metabolism can lead to novel strategies for treating, reducing or preventing boar taint. In addition, polymorphisms in these candidate genes may be useful as possible markers for low boar taint pigs.

### SUMMARY OF THE INVENTION

Broadly stated, the present invention relates to methods for determining the susceptibility of a pig to boar taint as well as to a method for reducing or preventing boar taint in male pigs or in breeding and selection of pigs.

The metabolism of skatole in pigs involves Phase I oxidation reactions carried out by cytochrome P450, and Phase II conjugation reactions carried out by glucuronyl transferases, sulfotransferases, in particular thermostable phenol sulfotransferase (SULT1A1) and glutathione transferases. According to the invention, applicants have found that many of the enzymes involved in these reactions are regulated by nuclear receptors, their concomitant ligands, inducers, repressers and the like. The nuclear receptors constitutive androstane receptor (CAR), pregnane X receptor (PXR) and farnesoid X receptor (FXR) have been investigated and found to have involvement in the metabolism of skatole and androstenone and thus are targets for interaction to reduce boar taint in pigs.

For example, the inventors show herein that compounds which activate these receptors (CAR, PXR, FXR) increase the expression of SULT2A1, and thus reduce boar taint. Expression of several genes involved in androstenone metabolism and skatole metabolism such as 3β-HSD, 3α-HSD, SULT2A1, UGT2B, CYP2A6, and CYP2E1 is affected by treatment with ligands for CAR, and treatment with inducers of PXR increased CYP2E1 activity, decreased CYP2A6 activity while also increasing production of two skatole metabolites. Pig CAR has several novel hormonal ligands that cause significant repressions of gene expression; these ligands include hormones in the Δ16 pathway: 5α-androsten-3β-ol, 5,16-androstadien-3β-ol, and the potent androgens 5α dihydrotestosterone (5α-DHT) and 5β-DHT. These compounds may repress the expression of genes involved in the metabolism of boar taint compounds. Thus the invention involves the manipulation of these nuclear receptors for the reduction of boar taint. This can include administration of inducers, ligands or removal of repressors and the like for pharmaceutical interaction to reduce boar taint, assays for differences in activities of these compounds to identify an animal's proclivity for boar taint, assaying for alternate gene forms which correlate with differences in boar taint, and even transgenic and genetic engineering protocols for these receptors with the outcome of reducing boar taint.

Accordingly, in one aspect, the present invention provides a method for assessing the ability of a pig to metabolise skatole or androstenone comprising (a) obtaining a sample from the pig and (b) detecting the levels of CAR, PXR and/or FXR in the sample wherein high levels of the same indicate that the pig is a good skatole or androstenone metabolizer. In another aspect, the present invention provides a method for determining the susceptibility of a male pig to boar taint comprising (a) obtaining a sample from the pig and (b) detecting the levels of CAR, PXR and/or FXR in the sample, wherein high levels of CAR, PXR and/or FXR indicates that the pig has a reduced susceptibility to developing boar taint. In a further aspect, the present invention provides a method for reducing boar taint comprising enhancing the activity of CAR, PXR and/or FXR in a pig. The activity of CAR, PXR and/or FXR can be enhanced by using substances which (a) increase the activity of CAR, PXR and/or FXR, such as ligands, inducers and the like or (b) induce or increase the expression of the CAR, PXR and/or FXR genes or by (c) removing repressors of these receptors.

The present invention also includes methods of identifying genetic markers which can be used in marker assisted breeding or in screening of animals for their proclivity towards boar taint comprising the following: a) screening the porcine CAR, FXR and/or PXR genes for polymorphisms, and b) correlating said polymorphisms in a given line, population or group with boar taint or with enzyme activity involved in skatole or androstenone metabolism, wherein a biologically significant difference from a baseline determination of the same represents a genetic marker for differences in boar taint.

The present invention also includes a method of screening for a substance that regulates skatole or androstenone metabolism in a pig. In one embodiment, the present invention provides a method for screening a substance that activates CAR, PXR and/or FXR activity or induces transcription and/or translation of a gene encoding CAR, PXR and/or FXR. The present invention also includes a pharmaceutical composition for use in treating boar taint comprising an effective amount of a substance which regulates skatole or androstenone metabolism in a pig and/or a pharmaceutical acceptable carrier, diluent or excipient.

The present invention further includes a method for producing pigs that have a lower incidence of boar taint comprising selecting pigs that express high levels of CAR, PXR and/or FXR, and breeding the selected pigs.

The invention also includes novel porcine CAR encoding sequences including several different isoforms which may be used in accordance with the invention. The invention also includes proteins, vectors, and genetic methods using peptides and proteins encoded by the CAR polynucleotides.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1: Effect of nuclear receptor ligands on SULT2A1 activity in porcine Leydig cells. Primary porcine Leydig cells were isolated from mature Yorkshire boars and the SULT2A1 activity was determined. Leydig cells were cultured in the presence of; CITCO (1 μM), TCPOBOP (250 nM), Phenobarbital (2 mM), Phenytoin (50 μM), Rifampicin (10 μM), PCN (10 μM), Dexamethasone (0.1 μM), Cholic Acid (100 μM), and Lithocolic Acid (100 μM) for 24 hours. Treatments are grouped according to the nuclear receptor affected: constitutive androstane receptor (CAR), pregnane X receptor (PXR), glucocorticoid receptor (GR) and farnesoid X receptor (FXR). Values are presented as means±SE of 3 independent experiments with significant differences indicated (*P<0.05).

FIG. 2: Determination of the effects of hormone and nuclear receptor agonists on CYP2E1 and CYP2A6 activities and on the production of 3MI metabolites. Following attachment of hepatocytes we treated cells with, isoproterenol (500 μM), testosterone (10 μM), estradiol (10 μM), estrone (10 μM), androstenol (10 μM), androstanol (10 μM), CITCO (1.0 μM), and rifampicin (10 μM) for 19 hours prior to the determination of (A, B) CYP2E1 and CYP2A6 activities as determined by PNP hydroxylase and COH assays respectively and (C, D) on the production of 3MI metabolites, HMOI and 3MOI in 3-week old (A, C) and adult (B, D) male hepatocytes. Each data point represents results obtained from 3 separate experiments run in triplicate. Values as percent of control are presented as means±SE, with significant differences (*P<0.05) within P450 activity or 3MI metabolite indicated.

FIG. 3. (A) Domains of CAR receptors and percent homology of hCAR and mCAR is compared to pgCAR at the nucleotide (nt) and protein (prn) levels. (B) The dimerization of NR1I to RXR to initiate target gene transcription is shown. Response element patterns are shown as DR-X, ER-X and IR-X, adapted from (Handschin and Meyer 2003)

FIG. 4. Isolation of porcine CAR (pgCAR) from DNase I treated liver cDNA Lane A, B, and C represent annealing temperatures of 62° C., 64° C., 66° C. respectively. Additional banding patterns show the presence of alternative spliced isoforms.

FIG. 5. Nucleotide alignment from BLAST search result identifies human CAR as most homologous DNA sequence to pgCAR.

FIG. 6. Dual Luciferase Reporter Assay: Pig, human [symbol] mouse [symbol] receptors were tested in a transient transfection assay against a panel of hormones and xenobiotics. Hormones and TCPOBOP were tested at 10 μM with the exception of CITCO at 1 μM. The effects of ligands are expressed as fold-change relative to vehicle (dimethylsulfoxide 1:2000) control for each receptor.

♦Indicates significant reporter gene fold change compared to DMSO A,B,C indicates if species response to ligands is significantly different, shared letters indicates not significantly different

FIG. 7. Dose response analysis of 0.01 uM/ml-10.0 uM/ml CITCO treatment on HepG2 cells transiently transformed by pgCAR and dual luciferase plasmids. Treatments>0.5 uM significantly activate pgCAR above the high basal levels in HepG2 cells.

FIG. 8. Full length pgCAR isoforms prior to digestion on a 1% agarose gel. B. RFLP NciI, NcoI double digest of full length pgCAR. The wild type splice variant 0 (SV0) produced a banding pattern of four fragments 353, 292, 265 and 148 bp in length, SV1-SV5 have altered migration or different number of fragments.

FIG. 9. Nucleotide alignment of the five alternative spliced isoforms of pgCAR. Sample 3087-1 is the wild type active form, all other isoform cause frameshifts.

FIG. 10. Splice variants (SV) of pgCAR delete (del) or insert (ins) sequence at exon junctions. SV0 the active form expresses all exons. SV1-5 alters the protein reading frame causing a loss of AF2 domain which is essential for nuclear translocation and gene regulation.

FIG. 11. Dose response analysis of CITCO ligand treatment in primary boar hepatocytes. Unlike HepG2 cells, primary hepatocytes have very low basal luciferase expression allowing for the detection of significant fold changes above no hormone control at lower doses.

FIG. 12. Dose response analysis of Phenytoin ligand treatment in primary boar hepatocytes.

FIG. 13. Dual luciferase assay of selected ligand in boar hepatocytes. Selected activators and repressors of pgCAR in HepG2 cells were tested in boar hepatocytes. As previously indicated, the extremely low basal reporter gene expression levels mask the inhibitory effects of 5β-DHT and 5α-DHT. Only reporter gene activations and not repressions are detectable in this cell model.

FIG. 14. Primers designed for all experiments are listed here

FIG. 15. Real time PCR primer efficiencies compared to B-actin housekeeping gene. Differences in primer efficiencies of>10% are not suitable for identifying expression differences between individual animals

FIG. 16. Boar hepatocytes treated with ligands for 8-10 hrs prior to RNA isolation and real time PCR analysis. B-actin was used as the housekeeping gene and DMSO (no hormone treatment) was used as the calibrator. For CYP2A6, CYP2E1, SULT1A1 and UGT2B the primer inefficiencies should not affect the resultant fold changes in this experiment since all hepatocyte treatments come from a single boar and analysis is compared to the no hormone DMSO control. This analysis should be valid since the calibrator is from the same pool of cells.

FIG. 17. Real time PCR gene expression from RNA extracted from the testis of High/Low androstenone boars. Results are expressed as a fold-change for each animal for 3β-HSD, SULT2A1, 3α-HSD, UGT2B, CYP2B6, and CAR. The lowest expressing animal for each gene is the calibrator.

### DETAILED DESCRIPTION OF THE INVENTION

### 1. Methods of Determining Susceptibility to Boar Taint

Accordingly, in one aspect the present invention provides a method for assessing the ability of a pig to metabolise skatole or androstenone comprising (a) obtaining a sample from the pig and (b) detecting the levels of CAR, PXR, and/or FXR in the sample wherein high levels of CAR, PXR, and/or FXR indicates that the pig is a good skatole or androstenone metabolizer. In another aspect, the present invention provides a method for determining the susceptibility of a male pig to developing boar taint comprising (a) obtaining a sample from the pig and (b) detecting the levels of CAR, PXR, and/or FXR in the sample, wherein low levels of CAR, PXR, and/or FXR indicates that the pig has an increased susceptibility to developing boar taint.

The sample from the pig can be any sample wherein levels of CAR, PXR, and/or FXR are correlated with levels of skatole or androstenone in fat and thus boar taint. In a preferred embodiment, the sample is a liver or testis sample or blood lymphocytes. The composition and activity of blood lymphocyte proteins, including CAR, PXR, and/or FXR, is closely related to that of the liver (Raucy et al., 1995; Yunjo et al., 1996). Levels of CAR, PXR, and/or FXR can be measured using techniques known in the art including Western blotting as described in Example 1. Levels of CAR, PXR, and/or FXR mRNA can also be measured by Northern analysis or quantitative PCR. Other methods include measuring the biological activity of the enzymes that are regulated by the receptors. For example, the activity of CAR, PXR, and/or FXR can be measured by assaying the reactions carried out by enzymes regulated by the receptors, for example assaying for N-nitrosodimethylamine demethylase activity, aniline hydroxylase activity or p-nitrophenol hydroxylase activity as described in Xu et al., 1994. Alternatively, the activity of CAR, PXR, and/or FXR can be measured by inhibiting the metabolism of skatole or androstenone using known CAR, PXR, and/or FXR inhibitors.

The term “high levels of CAR, PXR, and/or FXR” means that the sample contains the same or higher levels of CAR, PXR, and/or FXR than in a suitable control. Suitable controls include female pigs and male pigs that are known to have boar taint. When the control is a female pig “high levels of CAR, PXR, and/or FXR” means levels in the test pig are the same or higher than the control pig. When the control pig is a pig with boar taint, “high levels of CAR, PXR, and/or FXR” means levels in the test pig are higher, preferably about 2-3 times higher than the level in a pig with boar taint. More preferably, the levels in the test pig are higher, preferably 2-3 times higher than the average level of CAR, PXR, and/or FXR found in a group of pigs with boar taint. By “group” of pigs it is meant at least about 6 to about 10 male pigs.

### 2. Methods of Enhancing Skatole or Androstenone Metabolism

As hereinbefore mentioned, the present invention relates to a method for preventing boar taint by enhancing the metabolism of skatole or androstenone in a pig through the manipulation of nuclear receptors. For example, the inventors show herein that compounds which activate the receptors (CAR, PXR, FXR) increase the expression of SULT2A1, and thus reduce boar taint. Expression of several genes involved in androstenone metabolism and skatole metabolism such as 3β-HSD, 3α-HSD, SULT2A1, UGT2B, CYP2A6, and CYP2E1 is affected by treatment with ligands for CAR, and treatment with inducers of PXR increased CYP2E1 activity, decreased CYP2A6 activity while also increasing production of two skatole metabolites.

Accordingly, the present invention provides a method for reducing or preventing boar taint comprising enhancing the activity of CAR, PXR, and/or FXR in a pig. The activity of the CAR, PXR, and/or FXR enzyme can be enhanced by administering a substance (a) that activates or induces CAR, PXR, and/or FXR; or (b) a substance that induces or increases the expression of the CAR, PXR, and/or FXR gene. Substances that increase the activity of the CAR, PXR, and/or FXR or induce or increase the expression of the CAR, PXR, and/or FXR gene include substances such as ligands, or compounds which activate the receptors, or inducers. The activity of the CAR, PXR, and/or FXR may also be enhanced using gene therapy whereby a nucleic acid sequence encoding a CAR, PXR, and/or FXR enzyme is introduced into a pig, either ex-vivo or in vivo. A nucleic acid sequence encoding a CAR, PXR, and/or FXR enzyme may be obtained from GenBank or the novel sequences disclosed herein.

### 3. Screening Methods

As hereinbefore mentioned, the present invention provides a method of screening for a substance that affects skatole or androstenone metabolism by interacting with regulatory nuclear receptors involved in these metabolic pathways in a pig. Preferably, the substances affect the activity or expression of CAR, PXR, and/or FXR and are thus useful in reducing boar taint.

### Substances Which Activate CAR, FXR and PXR

In one aspect, the present invention provides a method of screening for a substance that enhances the activity of CAR, PXR, and/or FXR.

(a) CAR, PXR, and/or FXR

In one embodiment of the invention, a method is provided for screening for a substance that enhances skatole or androstenone metabolism in a pig by enhancing CAR, PXR, and/or FXR activity comprising the steps of:

(a) reacting a ligand or inducer of CAR, PXR, and/or FXR and CAR, PXR, and/or FXR, in the presence of a test substance, under conditions such that CAR, PXR, and/or FXR is capable of facilitating the transcription of genes encoding enzymes that metabolise boar taint compounds.

(b) assaying for unbound ligand, unreacted CAR, PXR, and/or FXR, or transcription of genes regulated by these receptors;

(c) comparing to controls to determine if the test substance selectively enhances CAR, PXR, and/or FXR activity and thereby is capable of enhancing skatole or androstenone metabolism in a pig.

Ligands or inducers of CAR, PXR, and/or FXR which may be used in the method of the invention, for example, include the compounds disclosed herein.

Levels of CAR, PXR, and/or FXR can be measured using techniques known in the art including Western blotting as described in Example 1. Levels of CAR, PXR, and/or FXR mRNA can also be measured by Northern analysis or quantitative PCR. Other methods include measuring the biological activity of the enzyme. For example, the activity of CAR, PXR, and/or FXR can be measured by estimating the effects on transcription of responsive genese as described in the examples. The CAR, PXR, and/or FXR, may be obtained from natural, recombinant, or commercial sources. Cells, particularly the cytoplasm or the nucleus expressing the enzymes may also be used in the method.

Conditions which permit the formation of a receptor ligand product may be selected having regard to factors such as the nature and amounts of the test substance and the ligand and the resultant transcription of regulated genes.

The ligand, receptor, unbound ligand, or unbound receptors may be isolated by conventional isolation techniques, for example, salting out, chromatography, electrophoresis, gel filtration, fractionation, absorption, polyacrylamide gel electrophoresis, agglutination, or combinations thereof. To facilitate the assay of the ligand receptor product, unbound ligand, or unbound receptor, antibody against the ligand receptor product or the ligand, or a labeled ligand, inducer or a labeled substance may be utilized. Antibodies, ligands, bound or unbound receptors, or the substance may be labeled with a detectable marker such as a radioactive label, antigens that are recognized by a specific labeled antibody, fluorescent compounds, enzymes, antibodies specific for a labeled antigen, and chemiluminescent compounds.

The ligand used in the method of the invention may be insolubilized. For example, it may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized enzyme, substrate, or substance may be prepared by reacting the material with a suitable insoluble carrier using known chemical or physical methods, for example, cyanogen bromide coupling.

Substances which Modulate Gene Expression

In another aspect, the present invention includes a method for screening for a substance that enhances skatole and/or androstenone metabolism by modulating the transcription or translation of nuclear receptor proteins involved in skatole and/or androstenone metabolism.

(a) CAR, PXR, and/or FXR

In one embodiment of the invention, a method is provided for screening for a substance that enhances skatole and/or androstenone metabolism by enhancing transcription and/or translation of the gene encoding CAR, PXR, and/or FXR comprising the steps of:

(a) culturing a host cell comprising a nucleic acid molecule containing a nucleic acid sequence encoding CAR, PXR, and/or FXR and the necessary elements for the transcription or translation of the nucleic acid sequence, and optionally a reporter gene, in the presence of a test substance; and

(b) comparing the level of expression of CAR, PXR, and/or FXR, or the expression of the protein encoded by the reporter gene with a control cell transfected with a nucleic acid molecule in the absence of the test substance.

A host cell for use in the method of the invention may be prepared by transfecting a suitable host with a nucleic acid molecule comprising a nucleic acid sequence encoding the appropriate enzyme. Suitable transcription and translation elements may be derived from a variety of sources, including bacterial, fungal, viral, mammalian, or insect genes. Selection of appropriate transcription and translation elements is dependent on the host cell chosen, and may be readily accomplished by one of ordinary skill in the art. Examples of such elements include: a transcriptional promoter and enhancer or RNA polymerase binding sequence, a ribosomal binding sequence, including a translation initiation signal. Additionally, depending on the host cell chosen and the vector employed, other genetic elements, such as an origin of replication, additional DNA restriction sites, enhancers, and sequences conferring inducibility of transcription may be incorporated into the expression vector. It will also be appreciated that the necessary transcription and translation elements may be supplied by the native gene of the enzyme and/or its flanking sequences.

Examples of reporter genes are genes encoding a protein such as β-galactosidase, chloramphenicol acetyltransferase, firefly luciferase, or an immunoglobulin or portion thereof such as the Fc portion of an immunoglobulin, preferably IgG. Transcription of the reporter gene is monitored by changes in the concentration of the reporter protein such as β-galactosidase, chloramphenicol acetyltransferase, or firefly luciferase. This makes it possible to visualize and assay for expression of the enzyme and in particular to determine the effect of a substance on expression of enzyme.

Suitable host cells include a wide variety of prokaryotic and eukaryotic host cells, including bacterial, mammalian, yeast or other fungi, viral, plant, or insect cells. Protocols for the transfection of host cells are well known in the art (see, Sambrook et al. Molecular Cloning A Laboratory Manual, 2nd edition, Cold Spring Harbor Laboratory Press, 1989, which is incorporated herein by reference). Host cells which are commercially available may also be used in the method of the invention. For example, the h2A3 and h2B6 cell lines available from Gentest Corporation are suitable for the screening methods of the invention.

### 4. Compositions

Substances which enhance skatole and/or androstenone metabolism described in detail herein or substances identified using the methods of the invention which selectively enhance CAR, PXR, and/or FXR (including antibodies or antisense sequences) may be incorporated into pharmaceutical compositions. Therefore, the invention provides a pharmaceutical composition for use in reducing boar taint comprising an effective amount of one or more substances which enhance skatole and/or androstenone metabolism and/or a pharmaceutically acceptable carrier, diluent, or excipient. In one embodiment, the present invention provides a pharmaceutical composition comprising an effective amount of the substance which is selected from the group consisting of

(a) a substance that increases the activity of the CAR, PXR, and/or FXR receptors;

(b) a substance that induces or increases the expression of the CAR, PXR, and/or FXR gene;

The substances for the present invention can be administered for oral, topical, rectal, parenteral, local, inhalant or intracerebral use. Preferably, the active substances are administered orally (in the food or drink) or as an injectable formulation.

In the methods of the present invention, the substances described in detail herein and identified using the method of the invention form the active ingredient, and are typically administered in admixture with suitable pharmaceutical diluents, excipients, or carriers suitably selected with respect to the intended form of administration, that is, oral tablets, capsules, elixirs, syrups and the like, consistent with conventional veterinary practices.

For example, for oral administration the active ingredients may be prepared in the form of a tablet or capsule for inclusion in the food or drink. In such a case, the active substances can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral active substances can be combined with any oral, non-toxic, pharmaceutically acceptable inert carrier such as ethanol, glycerol, water, and the like. Suitable binders, lubricants, disintegrating agents, and coloring agents can also be incorporated into the dosage form if desired or necessary. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, coaxes, and the like. Suitable lubricants used in these dosage forms include sodium oleate, sodium stearate, magnesium stearate, sodium benzoate, sodium acetate, sodium chloride, and the like. Examples of disintegrators include starch, methyl cellulose, agar, bentonite, xanthan gum, and the like.

Gelatin capsules may contain the active substance and powdered carriers, such as lactose, starch, cellulose derivatives, magnesium stearate, stearic acid, and the like. Similar carriers and diluents may be used to make compressed tablets. Tablets and capsules can be manufactured as sustained release products to provide for continuous release of active ingredients over a period of time. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and flavoring agents to increase acceptance.

Water, a suitable oil, saline, aqueous dextrose, and related sugar solutions and glycols such as propylene glycol or polyethylene glycols, may be used as carriers for parenteral solutions. Such solutions also preferably contain a water soluble salt of the active ingredient, suitable stabilizing agents, and if necessary, buffer substances. Suitable stabilizing agents include antioxidizing agents such as sodium bisulfate, sodium sulfite, or ascorbic acid, either alone or combined, citric acid and its salts and sodium EDTA. Parenteral solutions may also contain preservatives, such as benzalkonium chloride, methyl- or propyl-paraben, and chlorobutanol.

The substances described in detail herein and identified using the methods of the invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Substances described in detail herein and identified using the methods of the invention may also be coupled with soluble polymers which are targetable drug carriers. Examples of such polymers include polyvinylpyrrolidone, pyran copolymer, polyhydroxypropyl-methacrylamidephenol, polyhydroxyethyl-aspartamidephenol, or polyethyleneoxide-polylysine substituted with palmitoyl residues. The substances may also be coupled to biodegradable polymers useful in achieving controlled release of a drug. Suitable polymers include polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Suitable pharmaceutical carriers and methods of preparing pharmaceutical dosage forms are described in Remington's Pharmaceutical Sciences, Mack Publishing Company, a standard reference text in this field.

More than one substance described in detail herein or identified using the methods of the invention may be used to enhance metabolism of skatole or androstenone. In such cases the substances can be administered by any conventional means available for the use in conjunction with pharmaceuticals, either as individual separate dosage units administered simultaneously or concurrently, or in a physical combination of each component therapeutic agent in a single or combined dosage unit. The active agents can be administered alone, but are generally administered with a pharmaceutical carrier selected on the basis of the chosen route of administration and standard pharmaceutical practice as described herein.

### 5. Genetic Screening

The present invention further includes the identification of polynucleotide sequences, protein sequences, polymorphisms or other alternate gene forms in a pig in genes encoding CAR, PXR, and/or FXR, as described in detail herein. The identification of genes that encode these enzymes from pigs that are high skatole or androstenone metabolizers (and hence have a low incidence of low boar taint) can be used to develop lines of pigs that have a low incidence of boar taint. In addition, the identification of these genes can be used as markers for identifying pigs that are predisposed to having a low incidence of boar taint. According to the invention, applicants have identified the nucleotide sequence of porcine CAR as well as several alternate isoforms of the gene and the resulting amino acid sequences as well.

An embodiment of the invention is a method of identifying an allele of such genes that are associated with differences in skatole or androstenone metabolism and boar taint comprising obtaining a tissue or body fluid sample from an animal; amplifying DNA present in said sample comprising a region which includes a nuclear receptor gene involved in skatole and/or androstenone metabolism, preferably CAR, PXR or FXR; and detecting the presence of a polymorphic variant of said nucleotide sequences, wherein said variant is associated with a genetic predisposition either for or against boar taint in a particular line, population, species or group.

Another embodiment of the invention is a method of determining a genetic marker which may be used to identify and select animals based upon their proclivity to boar taint comprising obtaining a sample of tissue or body fluid from said animals, said sample comprising DNA; amplifying a region of DNA present in said sample, said region comprising a nucleotide sequence which encodes upon expression a nuclear receptor involved in skatole or androstenone metabolism, preferably CAR, FXR or PXR present in said sample from a first animal; determining the presence of a polymorphic allele present in said sample by comparison of said sample with a reference sample or sequence; correlating variability for boar taint in said animals with said polymorphic allele; so that said allele may be used as a genetic marker for the same in a given group, population, line or species.

Yet another embodiment of the invention is a method of identifying an animal for its propensity for boar taint, said method comprising obtaining a nucleic acid sample from said animal, and determining the presence of an allele characterized by a polymorphism in a nuclear receptor gene, preferably CAR, PXR, or FXR sequence present in said sample, or a polymorphism in linkage disequilibrium therewith, said genotype being one which is or has been shown to be significantly associated with a trait indicative of boat taint.

As used herein a “favorable boar taint trait” means a significant improvement (increase or decrease) in one of any measurable indicia of boar taint including compounds involved in skatole, or androstenone metabolism different from the mean of a given animal, group, line, species or population which has the alternate allele form, so that this information can be used in breeding to achieve a uniform group, line or species, or population which is optimized for these traits. This may include an increase in some traits or a decrease in others depending on the desired characteristics.

Methods for assaying for these traits generally comprises the steps 1) obtaining a biological sample from an animal; and 2) analyzing the genomic DNA or protein obtained in 1) to determine which allele(s) is/are present. Haplotype data which allows for a series of linked polymorphisms to be combined in a selection or identification protocol to maximize the benefits of each of these markers may also be used and are contemplated by this invention.

In another embodiment, the invention comprises a method for identifying further genetic markers in other linked genes for boar taint. Once a major effect gene has been identified, it is expected that other variations present in the same gene, allele or in sequences in useful linkage disequilibrium therewith may be used to identify similar effects on these traits without undue experimentation. The identification of other such genetic variation, once a major effect gene has been discovered, represents more than routine screening and optimization of parameters well known to those of skill in the art and is intended to be within the scope of this invention.

Differences between polymorphic forms of a specific DNA sequence may be detected in a variety of ways. For example, if the polymorphism is such that it creates or deletes a restriction enzyme site, such differences may be traced by using restriction enzymes that recognize specific DNA sequences. Restriction enzymes cut (digest) DNA at sites in their specific recognized sequence, resulting in a collection of fragments of the DNA. When a change exists in a DNA sequence that alters a sequence recognized by a restriction enzyme to one not recognized the fragments of DNA produced by restriction enzyme digestion of the region will be of different sizes. The various possible fragment sizes from a given region therefore depend on the precise sequence of DNA in the region. Variation in the fragments produced is termed “restriction fragment length polymorphism” (RFLP). The different sized-fragments reflecting variant DNA sequences can be visualized by separating the digested DNA according to its size on an agarose gel and visualizing the individual fragments by annealing to a labeled, e.g., radioactively or otherwise labeled, DNA “probe”.

PCR-RFLP, broadly speaking, is a technique that involves obtaining the DNA to be studied, amplifying the DNA, digesting the DNA with restriction endonucleases, separating the resulting fragments, and detecting the fragments of various genes. The use of PCR-RFLPs is the preferred method of detecting the polymorphisms, disclosed herein. However, since the use of RFLP analysis depends ultimately on polymorphisms and DNA restriction sites along the nucleic acid molecule, other methods of detecting the polymorphism can also be used and are contemplated in this invention. Such methods include ones that analyze the polymorphic gene product and detect polymorphisms by detecting the resulting differences in the gene product.

SNP markers may also be used in fine mapping and association analysis, as well as linkage analysis (see, e.g., Kruglyak (1997) Nature Genetics 17:21-24). Although a SNP may have limited information content, combinations of SNPs (which individually occur about every 100-300 bases) may yield informative haplotypes. SNP databases are available. Assay systems for determining SNPs include synthetic nucleotide arrays to which labeled, amplified DNA is hybridized (see, e.g., Lipshutz et al. (1999) Nature Genet. 21:2-24); single base primer extension methods (Pastinen et al. (1997) Genome Res. 7:606-614), mass spectroscopy on tagged beads, and solution assays in which allele-specific oligonucleotides are cleaved or joined at the position of the SNP allele, resulting in activation of a fluorescent reporter system (see, e.g., Landegren et al. (1998) Genome Res. 8:769-776).

The aim of association studies when used to discover genetic variation in genes associated with phenotypic traits is to identify particular genetic variants that correlate with the phenotype at the population level. Association at the population level may be used in the process of identifying a gene or DNA segment because it provides an indication that a particular marker is either a functional variant underlying the trait (i.e., a polymorphism that is directly involved in causing a particular trait) or is extremely close to the trait gene on a chromosome. When a marker analyzed for association with a phenotypic trait is a functional variant, association is the result of the direct effect of the genotype on the phenotypic outcome. When a marker being analyzed for association is an anonymous marker, the occurrence of association is the result of linkage disequilibrium between the marker and a functional variant.

There are a number of methods typically used in assessing genetic association as an indication of linkage disequilibrium, including case-control study of unrelated animals and methods using family-based controls. Although the case-control design is relatively simple, it is the most prone to identifying DNA variants that prove to be spuriously associated (i.e., association without linkage) with the trait. Spurious association can be due to the structure of the population studied rather than to linkage disequilibrium. Linkage analysis of such spuriously associated allelic variants, however, would not detect evidence of significant linkage because there would be no familial segregation of the variants. Therefore, putative association between a marker allele and a boar taint trait identified in a case-control study should be tested for evidence of linkage between the marker and the disease before a conclusion of probable linkage disequilibrium is made. Association tests that avoid some of the problems of the standard case-control study utilize family-based controls in which parental alleles or haplotypes not transmitted to affected offspring are used as controls.

In contrast to genetic linkage, which is a property of loci, genetic association is a property of alleles. Association analysis involves a determination of a correlation between a single, specific allele and a trait across a population, not only within individual groups. Thus, a particular allele found through an association study to be in linkage disequilibrium with a boar taint associated-allele can form the basis of a method of determining a predisposition to or the occurrence of the trait in any animal. Such methods would not involve a determination of phase of an allele and thus would not be limited in terms of the animals that may be screened in the method.

Methods for Identifying Genetic Markers Associated with Boar Taint

Also provided herein are methods of determining a genetic marker, which may be used to identify and select animals, based upon their boar taint traits. The methods include a step of testing a polymorphic marker on nuclear receptor genes, preferably CAR, PXR and/or FXR in association with boar taint traits. The testing may involve genotyping DNA from animals, and possibly be used as a genetic marker for the same in a given group, population or species, with respect to the polymorphic marker and analyzing the genotyping data for association with boar taint traits using methods described herein and/or known to those of skill in the art.

Any method of identifying the presence or absence of these polymorphisms may be used, including for example single-strand conformation polymorphism (SSCP) analysis, base excision sequence scanning (BESS), RFLP analysis, heteroduplex analysis, denaturing gradient gel electrophoresis, and temperature gradient electrophoresis, allelic PCR, ligase chain reaction direct sequencing, mini sequencing, nucleic acid hybridization, micro-array-type detection of a major effect gene or allele, or other linked sequences of the same. Also within the scope of the invention includes assaying for protein conformational or sequences changes, which occur in the presence of this polymorphism. The polymorphism may or may not be the causative mutation but will be indicative of the presence of this change and one may assay for the genetic or protein bases for the phenotypic difference. Based upon detection of these markers allele frequencies may be calculated for a given population to determine differences in allele frequencies between groups of animals, i.e. the use of quantitative genotyping. This will provide for the ability to select specific populations for associated traits.

In general, the polymorphisms used as genetic markers of the present invention find use in any method known in the art to demonstrate a statistically significant correlation between a genotype and a phenotype.

The invention therefore, comprises in one embodiment, a method of identifying an allele that is associated with boar taint traits. The invention also comprises methods of determining a genetic region or marker which may be used to identify and select animals based upon their boar taint predisposition. Yet another embodiment provides a method of identifying an animal for its propensity for boar taint traits.

Also provided herein are methods of detecting an association between a genotype and a phenotype, which may comprise the steps of a) genotyping at least one candidate nuclear receptor gene (preferably CAR, PXR, or FXR)-related marker in a trait positive population according to a genotyping method of the invention; b) genotyping the candidate gene-related marker in a control population according to a genotyping method of the invention; and c) determining whether a statistically or biologically useful, preferably significant, association exists between said genotype and said phenotype. In addition, the methods of detecting an association between a genotype and a phenotype of the invention encompass methods with any further limitation described in this disclosure, or those following, specified alone or in any combination. Preferably, the candidate gene-related marker is present in one or more of the nuclear receptor genes CAR, FXR or PXR. Each of said genotyping of steps a) and b) is performed separately on biological samples derived from each pig in said population or a subsample thereof. Preferably, the phenotype is a trait involving the boar taint, or concomitant skatole, or androstenone metabolism characteristics of an animal.

The invention described herein contemplates alternative approaches that can be employed to perform association studies: genome-wide association studies, candidate region association studies and candidate gene association studies. In a preferred embodiment, the markers of the present invention are used to perform candidate gene association studies. Further, the markers of the present invention may be incorporated in any map of genetic markers of the pig genome in order to perform genome-wide association studies. Methods to generate a high-density map of markers are well known to those of skill in the art. The markers of the present invention may further be incorporated in any map of a specific candidate region of the genome (a specific chromosome or a specific chromosomal segment for example).

Association studies are extremely valuable as they permit the analysis of sporadic or multifactor traits. Moreover, association studies represent a powerful method for fine-scale mapping enabling much finer mapping of trait causing alleles than linkage studies. Once a chromosome segment of interest has been identified, the presence of a candidate gene such as a candidate gene of the present invention, in the region of interest can provide a shortcut to the identification of the trait causing allele. Polymorphisms used as genetic markers of the present invention can be used to demonstrate that a candidate gene is associated with a trait. Such uses are specifically contemplated in the present invention and claims.

### Association Analysis

The general strategy to perform association studies using markers derived from a region carrying a candidate gene is to scan two groups of animals (case-control populations) in order to measure and statistically compare the allele frequencies of the markers of the present invention in both groups.

If a statistically significant association with a trait is identified for at least one or more of the analyzed markers, one can assume that: either the associated allele is directly responsible for causing the trait (the associated allele is the trait causing allele), or more likely the associated allele is in linkage disequilibrium with the trait causing allele. The specific characteristics of the associated allele with respect to the candidate gene function usually gives further insight into the relationship between the associated allele and the trait (causal or in linkage disequilibrium). If the evidence indicates that the associated allele within the candidate gene is most probably not the trait causing allele but is in linkage disequilibrium with the real trait causing allele, then the trait causing allele can be found by sequencing the vicinity of the associated marker.

Association studies are usually run in two successive steps. In a first phase, the frequencies of a reduced number of markers from the candidate gene are determined in the trait positive and trait negative populations. In a second phase of the analysis, the position of the genetic loci responsible for the given trait is further refined using a higher density of markers from the relevant region. However, if the candidate gene under study is relatively small in length, a single phase may be sufficient to establish significant associations.

### Testing for Association

Methods for determining the statistical significance of a correlation between a phenotype and a genotype, in this case an allele at a marker or a haplotype made up of such alleles, may be determined by any statistical test known in the art and is within any accepted threshold of statistical or biological significance being required. The application of particular methods and thresholds of significance are well within the skill of the ordinary practitioner of the art.

Testing for association is performed in one way by determining the frequency of a marker allele in case and control populations and comparing these frequencies with a statistical test to determine if there is a statistically significant difference in frequency which would indicate a correlation between the trait and the marker allele under study. Similarly, a haplotype analysis is performed by estimating the frequencies of all possible haplotypes for a given set of markers in case and control populations, and comparing these frequencies with a statistical test to determine if there is a statistically significant correlation between the haplotype and the phenotype (trait) under study. Any statistical tool useful to test for a statistically significant association between a genotype and a phenotype may be used and many exist. Preferably the statistical test employed is a chi-square test with one degree of freedom. A P-value is calculated (the P-value is the probability that a statistic as large or larger than the observed one would occur by chance). Other methods involve linear models and analysis of variance techniques.

The following is a general overview of techniques which can be used to assay for the polymorphisms of the invention.

In the present invention, a sample of genetic material is obtained from an animal. Samples can be obtained from blood, tissue, semen, etc. Generally, peripheral blood cells are used as the source, and the genetic material is DNA. A sufficient amount of cells are obtained to provide a sufficient amount of DNA for analysis. This amount will be known or readily determinable by those skilled in the art. The DNA is isolated from the blood cells by techniques known to those skilled in the art.

### Isolation and Amplification of Nucleic Acid

Samples of genomic DNA are isolated from any convenient source including saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W. H. Freeman & Co. New York (1992). Genomic DNA can also be isolated from cultured primary or secondary cell cultures or from transformed cell lines derived from any of the aforementioned tissue samples.

Samples of animal RNA can also be used. RNA can be isolated from tissues expressing the major effect gene of the invention as described in Sambrook et al., supra. RNA can be total cellular RNA, mRNA, poly A+RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, Kawasaki et al., Chapter 8 in PCR Technology,(1992) supra, and Berg et al., Hum. Genet. 85:655-658 (1990).

### PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, amniocytes from amniotic fluid, cultured chorionic villus cells, or the like are isolated by layering on sterile Ficoll-Hypaque gradient by standard procedures. Interphase cells are collected and washed three times in sterile phosphate buffered saline before DNA extraction. If testing DNA from peripheral blood lymphocytes, an osmotic shock (treatment of the pellet for 10 sec with distilled water) is suggested, followed by two additional washings if residual red blood cells are visible following the initial washes. This will prevent the inhibitory effect of the heme group carried by hemoglobin on the PCR reaction. If PCR testing is not performed immediately after sample collection, aliquots of 10⁶ cells can be pelleted in sterile Eppendorf tubes and the dry pellet frozen at −20° C. until use.

The cells are resuspended (10⁶ nucleated cells per 100 μl) in a buffer of 50 mM Tris-HCl (pH 8.3), 50 mM KCl, 1.5 mM MgCl₂, 0.5% Tween 20, 0.5% NP40 supplemented with 100 μg/ml of proteinase K. After incubating at 56° C. for 2 hr. the cells are heated to 95° C. for 10 min to inactivate the proteinase K and immediately moved to wet ice (snap-cool). If gross aggregates are present, another cycle of digestion in the same buffer should be undertaken. Ten μl of this extract is used for amplification.

When extracting DNA from tissues, e.g., chorionic villus cells or confluent cultured cells, the amount of the above mentioned buffer with proteinase K may vary according to the size of the tissue sample. The extract is incubated for 4-10 hrs at 50-60° C. and then at 95° C. for 10 minutes to inactivate the proteinase. During longer incubations, fresh proteinase K should be added after about 4 hr at the original concentration.

When the sample contains a small number of cells, extraction may be accomplished by methods as described in Higuchi, “Simple and Rapid Preparation of Samples for PCR”, in PCR Technology, Ehrlich, H. A. (ed.), Stockton Press, New York, which is incorporated herein by reference. PCR can be employed to amplify target regions in very small numbers of cells (1000-5000) derived from individual colonies from bone marrow and peripheral blood cultures. The cells in the sample are suspended in 20 μl of PCR lysis buffer (10 mM Tris-HCl (pH 8.3), 50 mM KCl, 2.5 mM MgCl₂, 0.1 mg/ml gelatin, 0.45% NP40, 0.45% Tween 20) and frozen until use. When PCR is to be performed, 0.6 μl of proteinase K (2 mg/ml) is added to the cells in the PCR lysis buffer. The sample is then heated to about 60° C. and incubated for 1 hr. Digestion is stopped through inactivation of the proteinase K by heating the samples to 95° C. for 10 min and then cooling on ice.

A relatively easy procedure for extracting DNA for PCR is a salting out procedure adapted from the method described by Miller et al., Nucleic Acids Res. 16:1215 (1988), which is incorporated herein by reference. Mononuclear cells are separated on a Ficoll-Hypaque gradient. The cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 150 μl of a 20% SDS solution are added to the cells and then incubated at 37° C. overnight. Rocking the tubes during incubation will improve the digestion of the sample. If the proteinase K digestion is incomplete after overnight incubation (fragments are still visible), an additional 50 μl of the 20 mg/ml proteinase K solution is mixed in the solution and incubated for another night at 37° C. on a gently rocking or rotating platform. Following adequate digestion, one ml of a 6 M NaCl solution is added to the sample and vigorously mixed. The resulting solution is centrifuged for 15 minutes at 3000 rpm. The pellet contains the precipitated cellular proteins, while the supernatant contains the DNA. The supernatant is removed to a 15 ml tube that contains 4 ml of isopropanol. The contents of the tube are mixed gently until the water and the alcohol phases have mixed and a white DNA precipitate has formed. The DNA precipitate is removed and dipped in a solution of 70% ethanol and gently mixed. The DNA precipitate is removed from the ethanol and air-dried. The precipitate is placed in distilled water and dissolved.

Kits for the extraction of high-molecular weight DNA for PCR include a Genomic Isolation Kit A.S.A.P. (Boehringer Mannheim, Indianapolis, Ind.), Genomic DNA Isolation System (GIBCO BRL, Gaithersburg, Md.), Elu-Quik DNA Purification Kit (Schleicher & Schuell, Keene, N. H.), DNA Extraction Kit (Stratagene, LaJolla, Calif.), TurboGen Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm. After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In a particularly useful embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn Hoffman-Berling, 1978, CSH-Quantitative Biology, 43:63-67; and Radding, 1982, Ann. Rev. Genetics 16:405-436, each of which is incorporated herein by reference).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art and are described in Gelfand, 1989, PCR Technology, supra.

### Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:12427-2448 (1989).

### Allele Specific Oligonucleotide Screening Methods

Further diagnostic screening methods employ the allele-specific oligonucleotide (ASO) screening methods, as described by Saiki et al., Nature 324:163-166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wild type allele.

### Ligase Mediated Allele Detection Method

Target regions of a test subject's DNA can be compared with target regions in unaffected and affected family members by ligase-mediated allele detection. See Landegren et al., Science 241:107-1080 (1988). Ligase may also be used to detect point mutations in the ligation amplification reaction described in Wu et al., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

### Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (TM). Melting domains are at least 20 base pairs in length, and may be up to several hundred base pairs in length.

Differentiation between alleles based on sequence specific melting domain differences can be assessed using polyacrylamide gel electrophoresis, as described in Chapter 7 of Erlich, ed., PCR Technology, Principles and Applications for DNA Amplification, W. H. Freeman and Co., New York (1992), the contents of which are hereby incorporated by reference.

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988), the contents of which are hereby incorporated by reference. The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

### Temperature Gradient Gel Electrophoresis

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis.

### Single-Strand Conformation Polymorphism Analysis

Target sequences or alleles at an particular locus can be differentiated using single-strand conformation polymorphism analysis, which identifies base differences by alteration in electrophoretic migration of single stranded PCR products, as described in Orita et al., Proc. Nat. Acad. Sci. 85:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

### Chemical or Enzymatic Cleavage of Mismatches

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from an animal and an affected family member may be used to generate mismatch free heterohybrid DNA duplexes. As used herein, “heterohybrid” means a DNA duplex strand comprising one strand of DNA from one animal, and a second DNA strand from another animal, usually an animal differing in the phenotype for the trait of interest. Positive selection for heterohybrids free of mismatches allows determination of small insertions, deletions or other polymorphisms that may be associated with polymorphisms.

### Non-Gel Systems

Other possible techniques include non-gel systems such as TaqMan™ (Perkin Elmer). In this system oligonucleotide PCR primers are designed that flank the mutation in question and allow PCR amplification of the region. A third oligonucleotide probe is then designed to hybridize to the region containing the base subject to change between different alleles of the gene. This probe is labeled with fluorescent dyes at both the 5′ and 3′ ends. These dyes are chosen such that while in this proximity to each other the fluorescence of one of them is quenched by the other and cannot be detected. Extension by Taq DNA polymerase from the PCR primer positioned 5′ on the template relative to the probe leads to the cleavage of the dye attached to the 5′ end of the annealed probe through the 5′ nuclease activity of the Taq DNA polymerase. This removes the quenching effect allowing detection of the fluorescence from the dye at the 3′ end of the probe. The discrimination between different DNA sequences arises through the fact that if the hybridization of the probe to the template molecule is not complete, i.e. there is a mismatch of some form; the cleavage of the dye does not take place. Thus only if the nucleotide sequence of the oligonucleotide probe is completely complimentary to the template molecule to which it is bound will quenching be removed. A reaction mix can contain two different probe sequences each designed against different alleles that might be present thus allowing the detection of both alleles in one reaction.

Yet another technique includes an Invader Assay which includes isothermic amplification that relies on a catalytic release of fluorescence. See Third Wave Technology at www.twt.com.

### Non-PCR Based DNA Diagnostics

The identification of a DNA sequence linked to an allele sequence can be made without an amplification step, based on polymorphisms including restriction fragment length polymorphisms in an animal and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with 32P or 35S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3,3′,5,5′-tetramethylbenzidine (TMB), fluorescein, and its derivatives, dansyl, umbelliferone and the like or with horse radish peroxidase, alkaline phosphatase and the like.

Hybridization probes include any nucleotide sequence capable of hybridizing to a porcine chromosome where one of the major effect genes resides, and thus defining a genetic marker linked to one of the major effect genes, including a restriction fragment length polymorphism, a hypervariable region, repetitive element, or a variable number tandem repeat. Hybridization probes can be any gene or a suitable analog. Further suitable hybridization probes include exon fragments or portions of cDNAs or genes known to map to the relevant region of the chromosome.

Preferred tandem repeat hybridization probes for use according to the present invention are those that recognize a small number of fragments at a specific locus at high stringency hybridization conditions, or that recognize a larger number of fragments at that locus when the stringency conditions are lowered.

One or more additional restriction enzymes and/or probes and/or primers can be used. Additional enzymes, constructed probes, and primers can be determined by routine experimentation by those of ordinary skill in the art and are intended to be within the scope of the invention.

Accordingly, the present invention provides a method for producing pigs which have a lower incidence of boar taint comprising selecting pigs that express high levels of CAR, PXR, and/or FXR; and breeding the selected pigs.

Transgenic pigs may also be prepared which produce high levels of CAR, PXR, and/or FXR. The transgenic pigs may be prepared using conventional techniques. For example, a recombinant molecule may be used to introduce a gene encoding CAR, PXR, and/or FXR Such recombinant constructs may be introduced into cells such as embryonic stem cells, by a technique such as transfection, electroporation, injection, etc. Cells which show high levels of CAR, PXR, and/or FXR may be identified for example by Southern Blotting, Northern Blotting, or by other methods known in the art. Such cells may then be fused to embryonic stem cells to generate transgenic animals. Germline transmission of the mutation may be achieved by, for example, aggregating the embryonic stem cells with early stage embryos, such as 8 cell embryos, transferring the resulting blastocysts into recipient females in vitro, and generating germline transmission of the resulting aggregation chimeras. Such a transgenic pig may be mated with pigs having a similar phenotype i.e. producing high levels of CAR, PXR, and/or FXR to produce animals having a low incidence of boar taint.

According to the invention, the applicants have also identified novel sequence.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison; in this case, the Reference sequences. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence, a gap penalty is typically introduced and is subtracted from the number of matches.

Methods of alignment of sequences for comparison are well known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/).

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e)(I) The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

These programs and algorithms can ascertain the analogy of a particular polymorphism in a target gene to those disclosed herein. It is expected that this polymorphism will exist in other animals and use of the same in other animals than disclosed herein involved no more than routine optimization of parameters using the teachings herein.

The invention is intended to include the disclosed sequences as well as all conservatively modified variants thereof. The terms CAR, FXR and/or PXR as used herein shall be interpreted to include conservatively modified variants. The term “conservatively modified variants” applies to both amino acid and nucleic acid sequences. With respect to particular nucleic acid sequences, conservatively modified variants refer to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid. One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7, or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80%, or 90% of the native protein for its native substrate. Conservative substitution tables providing functionally similar amino acids are well known in the art.

Conservative substitutions of encoded amino acids include, for example, amino acids that belong within the following groups: (1) non-polar amino acids (Gly, Ala, Val, Leu, and Ile); (2) polar neutral amino acids (Cys, Met, Ser, Thr, Asn, and Gln); (3) polar acidic amino acids (Asp and Glu); (4) polar basic amino acids (Lys, Arg and His); and (5) aromatic amino acids (Phe, Trp, Tyr, and His).

Those of ordinary skill in the art will recognize that some substitution will not alter the activity of the polypeptide to an extent that the character or nature of the polypeptide is substantially altered. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics, e.g., with boar taint-like characteristics. When it is desired to alter the amino acid sequence of a polypeptide to create an equivalent, or a variant or portion of a polypeptide of the invention, one skilled in the art will typically change one or more of the codons of the encoding DNA sequence according to Table 1 (See infra). For example, certain amino acids may be substituted for other amino acids in a protein structure without appreciable loss of activity. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences, which encode said peptides without appreciable loss of their biological utility or activity. A degenerate codon means that a different three letter codon is used to specify the same amino acid. For example, it is well known in the art that the following RNA codons (and therefore, the corresponding DNA codons, with a T substituted for a U) can be used interchangeably to code for each specific amino acid:

TABLE 1

| Amino Acids | Codons |
|---|---|
| Phenylalanine (Phe or F) | UUU, UUC, UUA or UUG |
| Leucine (Leu or L) | CUU, CUC, CUA or CUG |
| Isoleucine (Ile or I) | AUU, AUC or AUA |
| Methionine (Met or M) | AUG |
| Valine (Val or V) | GUU, GUC, GUA, GUG |
| Serine (Ser or S) | AGU or AGC |
| Proline (Pro or P) | CCU, CCC, CCA, CCG |
| Threonine (Thr or T) | ACU, ACC, ACA, ACG |
| Alanine (Ala or A) | GCU, GCG, GCA, GCC |
| Tryptophan (Trp) | UGG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | AGA or AGG |
| Glycine (Gly or G) | GGU or GGC or GGA or GGG |
| Termination codon | UAA, UAG or UGA |

The following non-limiting examples are illustrative of the present invention:

### EXAMPLES

### Example 1

### The Role of Nuclear Receptors in SULT2A1 Expression and Sulfoconjugation of Androstenone

### Introduction

Hydroxysteroid sulfotransferase (SULT2A1) is responsible for sulfoconjugating the 16-androstene steroids (Sinclair & Squires 2005), which have pheromonal properties and are the most abundant steroids produced by the boar testes. The accumulation of high levels of the 16-androstene steroid, 5α-androstenone, in adipose tissue produces an unpleasant odor when the meat from intact males is cooked (Patterson 1968). Our previous studies have indicated that increased levels of sulfoconjugated 16-androstene steroids present in the systemic circulation are associated with a reduction in the accumulation of 5α-androstenone in adipose tissue (Sinclair & Squires 2005). In humans, SULT2A1 activity varies up to 5-fold among individuals (Weinshilboum & Aksoy 1994), suggesting that genetic polymorphisms may be involved in regulating SULT2A1 activity. A number of studies reported single nucleotide polymorphisms (SNPs) within the human Sult2A1 gene, (Igaz et al. 2002), some of which have resulted in reduced levels of both enzyme activity and protein (Thomae et al. 2002b). Furthermore, research in both human and rodent SULT2A1 regulation has revealed that this gene may be controlled by various nuclear receptors including the constitutive androstane receptor (CAR) (Saini et al. 2004), the pregnane X receptor (PXR) (Kliewer et al. 1998, Duanmu et al. 2002, Echchgadda et al. 2004), and the famesoid X receptor (FXR) (Makishima et al. 1999, Song et al. 2001). However, the involvement of nuclear receptors in the regulation of porcine SULT21 has not yet been shown.

### Materials and Methods

### Reagents

Sep-Pak C₁₈ solid-phase chromatography cartridges were purchased from Waters Ltd. (Mississauga, ON, Canada). HANKS Balanced Salt Solution (HBSS) was obtained from Invitrogen Life Technologies, (Burlington, ON, Canada). Radio labeled [³H] DHEA and DHEAS (approximately 0.01-0.05 μCi/nmol) were obtained from ICN Diagnostics (Montreal, QC, Canada). 5α-androstenone was obtained from Steraloids Inc. (Newport, R.I., USA) and 6-(4-chlorophenyl)imidazo[2,1-b][1,3]thiazole-5-carbaldehyde O-(3,4-dichlorobenzyl)oxime (CITCO) was obtained from Biomol Research Laboratories Inc. (Plymouth Meeting, Pa., USA). Pregnenolone 16α-carbonitrile (PCN), 1,4-bis-[2-(3,5-dichloropyridyl-oxy)]benzene; 3,3′5,5′-tetrachloro-1,4-bis(pyridyl-oxy)benzene (TCPOBOP) and all other reagents were obtained from Sigma-Aldrich Ltd. (Mississauga, ON, Canada).

### Tissue Samples

Mature Yorkshire boars of 175±6 days obtained from the Arkell Swine Research Station at the University of Guelph, Guelph, ON were used for the isolation of Leydig cells. After slaughter, the testes were immediately removed and transported to the laboratory within five minutes. Testicular tissue was sliced into pieces of 1 cm²×2-3 mm thickness and 100 g of tissue was incubated for 20 minutes in a shaking water bath at 37° C. with 1 mg/ml collagenase (type 1A), 50 μg/ml DNase and 50 μg/ml trypsin inhibitor in 250 ml of TC 199 Media containing 1 g/L bovine serum albumin and 0.1 g/L L-glutamine. Preparations of purified Leydig cells were obtained by layering the collagenase—dispersed testicular cells onto discontinuous Percoll gradients supplemented with HBSS. The preparations were then centrifuged at 1500×g for 15 minutes at 4° C. and the cells present at the 40-60% interface were collected as outlined previously (Raeside & Renaud 1983). Cell viability was determined by trypan blue exclusion. The typical viability of Leydig cells after this procedure was greater than 90%.

### Steroid Extraction and Analysis

Conjugated steroids were separated from unconjugated steroids using methanol primed Sep-Pak C₈ solid-phase chromatography cartridges (Raeside & Christie 1997). Sulfoconjugated steroids were hydrolyzed by incubating the conjugate fraction overnight in trifluoroacetic acid/ethyl acetate (1/100 v/v) at 45° C. The hydrolyzed steroids were then purified by Sep-Pak C₁₈ solid-phase chromatography.

### Identification of Nuclear Receptors Involved in Regulating SULT2A1

To further understand the molecular mechanisms that control SULT2A1 gene transcription, various nuclear receptor ligands were evaluated for their ability to induce SULT2A1 activity in Leydig cells. Aliquots of re-suspended Leydig cells (1×10⁶ cells/ml) in TC 199 Media mixture were plated in 24 well plates. The Leydig cells were then treated with various nuclear receptor ligands as outlined in the figure legends in less than 0.5% DMSO vehicle and incubated at 37° C. in 95% air and 5% CO₂ for 24 hours.

After the 24 h incubation period, the media was aspirated and approximately 10,000 cpm of [³H] DHEA (0.01-0.05 μCi/nmol) was added to each well in a final volume of 1 ml of TC 199 Media. The cells were incubated for 4 h and the media was removed and immediately extracted by Sep-Pak C₁₈ solid-phase chromatography, as described above. Sulfotransferase activity was assayed by measuring the conversion of [³H] DHEA (free) to [³H] DHEAS (conjugate) fractions by liquid scintillation counting. The results from the different Leydig cell preparations were normalized as a percentage of the average results from the control incubations from each cell preparation.

### Statistical Analyses

Differences in SULT2A1 activity following treatment with nuclear receptor ligands were analyzed by ANOVA using the Dunnet post-hoc test (SAS 8.2, SAS Inst. Inc.).

### Results and Discussion

### Identification of Nuclear Receptors Involved in Regulating SULT2A1

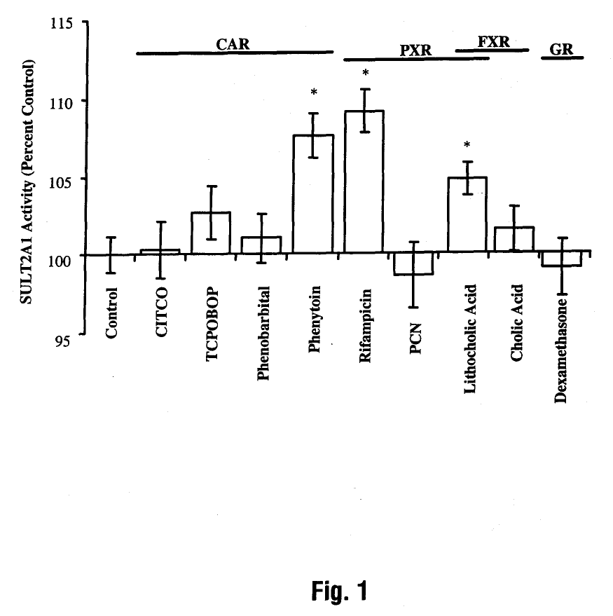

To further understand the transcriptional regulation of testicular SULT2A1, primary isolated Leydig cells were incubated with various nuclear receptor ligands for 24 hours prior to the determination of SULT2A1 activity. As seen in FIG. 1, SULT2A1 activity was increased by treatment with some of the ligands to CAR, PXR and FXR. Of the CAR inducers, only phenytoin resulted in a significant increase in SULT2A1 activity (P<0.05). Rifampicin, a ligand of human and pig PXR (Moore et al. 2002) significantly increased (P<0.05) SULT2A1 activity. The glucocorticoid receptor (GR) ligand dexamethasone did not affect SULT2A1 activity, whereas treatment with the FXR/PXR ligand lithocolic acid resulted in an increase in SULT2A1 activity (P<0.05).

The extent to which 5α-androstenone accumulates in fat is influenced by the amount of unconjugated steroid that is present in the circulation. Differences in the ability to sulfoconjugate 5α-androstenone will affect the level of unconjugated steroid that is available to accumulate in fat. The levels of sulfoconjugated 16-androstene steroids present in the circulation are a result of the balance between the capacity for testicular steroidogenesis, sulfoconjugation, and metabolic clearance. Previous results show that the concentration of sulfoconjugated 5α-androstenone in the peripheral plasma is correlated with testicular SULT2A1 activity (r=0.66) and to a lesser extent to hepatic SULT2A1 activity (r=0.13). Furthermore, we show that treatment with ligands for the nuclear receptors CAR, PXR and possibly FXR can stimulate SULT2A1 activity in isolated Leydig cells. Together these results further clarify the magnitude of testicular sulfoconjugation of androstenone and the role of SULT2A1, and suggest possible control of porcine SULT2A1 expression by nuclear receptors.

The molecular mechanisms that control SULT2A1 gene transcription have not been fully characterized. Chenodeoxycholic acid has been demonstrated to be a strong inducer of the rat SULT2A1 gene (Makishima et al. 1999). This inducing effect is controlled in part by the bile acid-activated FXR (Song et al. 2001). Similarly, human SULT2A1 has been shown to be induced in response to various ligands for the PXR (Kliewer et al. 1998, Duanmu et al. 2002, Echchgadda et al. 2004). More recently, it has been determined that the CAR is also potentially involved in rodent SULT2A1 regulation (Saini et al. 2004). The results of this study suggest that the recently identified human CAR activator phenytoin (Jackson et al. 2004) resulted in a significant increase in pig SULT2A1 activity, whereas CITCO (Maglich et al. 2003) and CAR agonists TCBOPOP and phenobarbital did not affect SULT2A1 activity. While these classical inducers are known as potent rodent inducers, their influence on human gene expression is often absent or attenuated in comparison to rodents (Moore et al. 2002). Similarly, the treatment of Leydig cells with the PXR ligands rifampicin and PCN demonstrated that only rifampicin resulted in a significant increase in SULT2A1 activity. This observation is consistent with the results of Moore et al. (2002) who demonstrated in a reporter assay system that rifampicin is a potent activator of pig PXR. However, PCN, a PXR inducer in mouse, rabbit, and monkey, does not activate either human or pig PXR. Furthermore, dexamethasone, which can affect CAR and PXR both directly and indirectly (Pascussi et al. 2000ab) did not increase SULT2A1 activity of Leydig cells at 10⁻⁷ M within the 24 hour treatment. In comparison to these exogenous ligands, lithocholic acid, a bile acid that acts through the FXR, induced pig SULT2A1 activity. However, Moore et al. (2002) demonstrated that lithocholic acid, but not cholic acid, may also activate porcine PXR. Together, these data suggest the importance of CAR, PXR, and potentially FXR in the regulation of SULT2A1 activity. This does not exclude the involvement of other orphan receptors in the regulation of pig SULT2A1 not investigated here. For instance, both steroidogenic factor 1 (SF1) and GATA-6 have been shown to regulate the transcription of human Sult2A1 (Saner et al., 2005). Furthermore, this study also highlights the similarity in responsiveness of nuclear receptors of human and pig compared to rodent species. Differences in the level of gene transcription of Sult2A1 may be due to individual differences in the activation of these receptors or differences in the proximal promoter region of the gene that contain the recognition sequences for these transcription factors. However, the 5′-proximal promoter region of porcine Sult2A1 gene has not yet been characterized

### REFERENCES

Duanmu Z, Locke D, Smigelski J, Wu W, Dahn M S, Falany C N, Kocarek T A & Runge-Morris M 2002 Effects of Dexamethasone on Aryl (SULT1A1)—and Hydroxysteroid (SULT2A1)—Sulfotransferase Gene Expression in Primary Cultured Human Hepatocytes. Drug Metab Dispos 30 997-1004.
Echchgadda I, Song C S, Oh T S, Cho S H, Rivera O J & Chatterjee B 2004 Gene Regulation for the Senescence Marker Protein DHEA-Sulfotransferase by the Xenobiotic-Activated Nuclear Pregnane X Receptor (PXR). Mech Ageing Dev 125 733-45.
Igaz P, Pap E, Patocs A, Falus A, Tulassay Z & Racz K 2002 Genomics of Steroid Hormones: in Silico Analysis of Nucleotide Sequence Variants (Polymorphisms) of the Enzymes Involved in the Biosynthesis and Metabolism of Steroid Hormones. J Steroid Biochem Mol Biol 82 359-67.
Jackson J P, Ferguson S S, Moore R, Negishi M & Goldstein J A 2004 The Constitutive Active/Androstane Receptor Regulates Phenytoin Induction of Cyp2c29. Mol Pharmacol 65 1397-404.
Kliewer S A, Moore J T, Wade L, Staudinger J L, Watson M A, Jones S A, McKee D D, Oliver B B, Willson T M, Zetterstrom R H, Perlmann T & Lehmann J M 1998 An Orphan Nuclear Receptor Activated by Pregnanes Defines a Novel Steroid Signaling Pathway. Cell 92 73-82.
Maglich J M, Parks D J, Moore L B, Collins J L, Goodwin B, Billin A N, Stoltz C A, Kliewer S A, Lambert M H, Will son T M & Moore J T 2003 Identification of a Novel Human Constitutive Androstane Receptor (CAR) Agonist and Its Use in the Identification of CAR Target Genes. J Biol Chem 278 17277-83.
Makishima M, Okamoto A Y, Repa J J, Tu H, Learned R M, Luk A, Hull M V, Lustig K D, Mangelsdorf D J & Shan B 1999 Identification of a Nuclear Receptor for Bile Acids. Science 284 1362-5.
Moore L B, Maglich J M, McKee D D, Wisely B, Willson T M, Kliewer S A, Lambert M H & Moore J T 2002 Pregnane X Receptor (PXR), Constitutive Androstane Receptor (CAR), and Benzoate X Receptor (BXR) Define Three Pharmacologically Distinct Classes of Nuclear Receptors. Mol Endocrinol 16 977-86.
Pascussi J M, Drocourt L, Fabre J M, Maurel P & Vilarem M J 2000a Dexamethasone Induces Pregnane X Receptor and Retinoid X Receptor-Alpha Expression in Human Hepatocytes: Synergistic Increase of CYP3A4 Induction by Pregnane X Receptor Activators. Mol Pharmacol 58 361-72.
Pascussi J M, Gerbal-Chaloin S, Fabre J M, Maurel P & Vilarem M J 2000b Dexamethasone Enhances Constitutive Androstane Receptor Expression in Human Hepatocytes: Consequences on Cytochrome P450 Gene Regulation. Mol Pharmacol 58 1441-50.
Patterson R L S 1968 5a-Androst-16-Ene-3-One:-Compound Responsible for Taint in Boar Fat. J Sci Food Ag 19 31-38.
Raeside J I & Christie H L 1997 Estrogen Concentrations in Semen of the Stallion. Anim Reprod Sci 48 293-300.
Raeside J I & Renaud R L 1983 Estrogen and Androgen Production by Purified Leydig Cells of Mature Boars. Biol Reprod 28 727-33.
Saini S P, Sonoda J, Xu L, Toma D, Uppal H, Mu Y, Ren S, Moore D D, Evans R M & Xie W 2004 A Novel Constitutive Androstane Receptor-Mediated and CYP3A-Independent Pathway of Bile Acid Detoxification. Mol Pharmacol 65 292-300.
Saner K J, Suzuki T, Sasano H, Pizzey J, Ho C, Strauss J F, Carr B R, Rainey W E 2005 Steroid sulfotransferase 2A1 gene transcription is regulated by steroidogenic factor 1 and GATA-6 in the human adrenal. Mol Endocrinol. 19:184-97.
Sinclair P A, Hancock S, Gilmore W J & Squires E L 2005 Metabolism of the 16-Androstene Steroids in Primary Cultured Porcine Hepatocytes. J Steroid Biochem 96 79-87.
Sinclair P A & Squires E J 2005 Testicular Sulfoconjugation of the 16-Androstene Steroids by Hydroxysteroid Sulfotransferase: Its Effect on the Concentrations of 5alpha-Androstenone in Plasma and Fat of the Mature Domestic Boar. J Anim Sci 83 358-65.
Song C S, Echchgadda I, Baek B S, Ahn S C, Oh T, Roy A K & Chatterjee B 2001 Dehydroepiandrosterone Sulfotransferase Gene Induction by Bile Acid Activated Farnesoid X Receptor. J Biol Chem 276 42549-56.
Thomae B A, Eckloff B W, Freimuth R R, Wieben E D & Weinshilboum R M 2002a Human Sulfotransferase SULT2A1 Pharmacogenetics: Genotype-to-Phenotype Studies. Pharmacogenomics J 2 48-56.
Thomae B A, Eckloff B W, Freimuth R R, Wieben E D & Weinshilboum R M 2002b Human Sulfotransferase SULT2A1 Pharmacogenetics: Genotype-to-Phenotype Studies. Pharmacogenomics J 2 48-56.
Weinshilboum R & Aksoy I 1994 Sulfation Pharmacogenetics in Humans. Chem Biol Interact 92 233-46.

### Example 2

### Role of Nuclear Receptors on CYP2A6 and CYP2E1 Activity and the Metabolism of Skatole in Pig Liver

### Abstract

Hepatic cytochrome P450 (CYP) 2E1 and CYP2A6 have been shown to play a role in the metabolism of skatole in pigs. This study investigates the role of CYP2E1 and CYP2A6 enzymes in hepatocytes from adult and 3-week old intact male pigs. We treated hepatocytes with isoproterenol, testosterone, estradiol, estrone, androstenol and androstanol, and with a constitutively activated androstane receptor (CAR) and pregnane X receptor (PXR) agonists, 6-(4-chlorophenyl) imidazo[2,1-b][1,3]thiazole-5-carbaldehyde O-(3,4-dichlorobenzyl)oxime (CITCO) and rifampicin respectively. We found that hepatocytes from 3-week old males treated with isoproterenol and with rifampicin caused an alteration in CYP2E1 and CYP2A6 activities respectively, while also increasing the metabolism of 3MI metabolites HMOI and 3MOI. In comparison, only isoproterenol was able to increase HMOI production in adult male hepatocytes. Hormones did not affect CYP2E1 and CYP2A6 activities in 3-week old male hepatocytes; however, treatment of adult male hepatocytes with estrone resulted in an increase in production of HMOI without a significant increase in CYP2E1 or CYP2A6 activities. Together these data show that CYP2E1 and CYP2A6 are differentially involved in the metabolism of 3MI within the developing pig and that other enzymes including different P450s may also be involved in the metabolism of 3MI, which maybe influenced by levels of estrone.

### Introduction

Hepatic cytochrome P450 (CYP) 2E1 and CYP2A6 have been shown to play a role in the metabolism of 3-methylindole (3MI, skatole) in pigs (Babol et al, 1998, Friis, 1995; Squires and Lundström, 1997; Diaz and Squires, 2000a). 3MI is produced in the hindgut of pigs by the breakdown of tryptophan (Yost, 1989). Due to its lipophilicity, 3MI accumulates in the fat of pigs and produces an off odor associated with boar meat when cooked, thus rendering the meat unpalatable (Bonneau, 1997). The accumulation of 3MI in fat increases with the onset of puberty, which is associated with increases in testes-derived hormones, including the pheromone androstenone (Zamaratskaia et al., 2004). As a result, males destined for meat production are castrated. The hepatic metabolism of 3MI into excretable forms (Claus et al., 1994; Squires and Bonneau, 2004) is important in the clearance of 3MI (Friis, 1992). Therefore, identification of factors that control 3MI liver mediated metabolism through CYP2E1 and CYP2A6 is required to better understand the underlying mechanisms in 3MI accumulation.

As castration reduces taint in boars (Babol et al, 1996), it stands to reason that a testicular factor is most likely a key regulator of metabolism in the pig. Boars secrete large amounts of estrogens from the testes, with conjugated estrogens in the range of the typical “male” hormones, including testosterone and 5α-androstenone (Claus and Hoffmann, 1980). Zamaratskaia et al. (2005) have recently reported a relationship between adipose free estrone and 3MI levels. Moran et al. (2002) reported a 50% decrease in testicular microsomal P450 concentrations when testes development was suppressed, suggesting there is testicular regulation of P450 levels in the neonatal pig. A correlation between testis-derived androstenone and a reduction in 3MI metabolism in porcine liver microsomes has been observed (Babol et al., 1999). Androstenone has also been reported by Doran et al. (2002) to block the 3MI-induced increase in CYP2E1 protein levels in cultured porcine hepatocytes.

The co-ordination of gene expression by hormones occurs via nuclear receptors. Two recently identified nuclear receptors, CAR and PXR, can be activated by various endobiotics to regulate the expression of genes encoding enzymes involved in liver metabolism (Moore et al., 2002; Maglich et al., 2002). The androstenone derivatives androstenol and androstanol have been shown to repress the constitutive activity of mouse CAR by promoting co-activator release from the ligand-binding domain (Forman et al., 1998). Other testicular hormones have also been reported in the mouse to affect the activity of CAR, including a decrease in activity by testosterone and increases in activity by estradiol and estrone (Kawamoto et al., 2000). Exogenous compounds have also been shown to modulate the expression of nuclear receptors and expression of P450s. For instance, Maglich et al. (2003) has demonstrated in recombinant expression systems that CITCO, an agonist of human CAR, causes the induction of various P450s in primary human hepatocytes. Rifampicin has been shown to activate human PXR (Desai et al., 2002; Chen et al., 2004) and pig PXR (Moore et al., 2002, 2003). In addition, PXR activation has been shown to turn on a battery of genes associated with metabolism, including the induction of a diverse group of P450s in primary cultured hepatocytes. (Reinach et al., 1999; Maglich et al., 2002).

This study was undertaken to determine the influence of inducers on CYP2E1 and CYP2A6 mediated 3MI metabolism in hepatocytes from 3-week old and adult male pigs.

### Materials and Method

### Chemicals

Hormone treatments androstenol and androstanol were obtained from Steraloids Inc. (Newport, R.I., USA). HANKS Balanced Salt Solution (HBSS) was obtained from Invitrogen Life Technologies, (Burlington, ON, Canada) and CITCO was obtained from Biomol Research Laboratories Inc. (Plymouth Meeting, Pa., USA). The 3MI metabolite standards 3-methyloxindole (3MOI) and 3-hydroxy-3-methyloxindole (HMOI) were a gift provided by Dr. G. S. Yost, Department of Pharmacology and Toxicology, University of Utah (Salt Lake City, Utah). Type I collagenase (activity 274 U/mg) was purchased from Worthington Biochemical Corporation (Lakewood, N.J., USA). Iletin® regular insulin (beef and pork) was purchased from Eli Lilly Company (Indianapolis, Ind., USA). Organic solvents were of HPLC grade from Fisher Scientific (Toronto, ON, Canada). 3-Methylindole, indole-3-carbinole, 2-aminoacetophenone, isoproterenol, testosterone, estradiol, estrone, rifampicin, and all remaining reagents were acquired from Sigma (Oakville, ON, Canada).

### Research Animals

All animals used for these experiments were obtained from Arkell Swine Research facility of the University of Guelph and used in accordance with the guidelines of the Canadian Council on Animal Care. Either uncastrated Yorkshire males at 21±1 days of age (3-weeks old) or mature male Yorkshire boars of 175±6 days of age pigs were used for hepatocytes isolation.

### Liver Perfusion and Hepatocyte Culture

Hepatocytes were prepared by a modification of a collagenase perfusion method described previously (Sinclair et al., 2005). Briefly, one hepatic lobe was immediately catheterized by selecting the largest vessel at the cut end. Blood was then flushed from the liver by perfusion with 25 mL/min of medium containing 10 mM hydroxyethyl piperazine ethane (HEPES), 1 mM EGTA and HBSS (pH 7.4) delivered at 37° C. The lobe was then perfused for 5 minutes with the same medium without EGTA and then with William's E Media (pH 7.4) containing 10 mM HEPES and 0.7 mg/mL collagenase type 1 for 10 to 25 minutes until the hepatocytes appeared to be fully dissociated. Length of time for collagenase digestion was longer in the mature pigs due to increased amount of collagenous connective tissue.

All remaining experimental procedures were carried out under sterile conditions. The lobe was gently minced with a scalpel to disassociate cells in attachment medium containing 10 mM HEPES, 10% (v/v) fetal bovine serum, 1% (v/v) penicillin-streptomycin and 50 Units/L insulin in William's E media (pH 7.4), at 4° C. Cells were filtered through gauze and centrifuged at 15×g for 3 minutes. The cell pellet was washed in attachment media and cell viability was determined by trypan blue exclusion. Hepatocytes were only used if viability was greater than 85%. Hepatocytes were then plated on Primaria™ 60 mm tissue culture dishes (VWR International Ltd., Mississauga, ON) at a density of 2.25×10⁶ cells in 3.0 mL attachment media, and kept in a incubator at 37° C. supplied with 95% air and 5% CO₂. After 4 hours to allow for attachment of the cells, the medium was replaced with serum-free medium that contained 10 mM HEPES, 10 mM pyruvate, 0.35 mM proline, 1% (v/v) penicillin-streptomycin and 50 Units/L insulin in William's E media (pH 7.4) with treatments. For inducer experiments, hepatocytes were treated for 19 hours prior to the determination of P450 activities and 3MI metabolism.

### P450 Activity Assays

To determine the rate of p-nitrophenol (PNP) hydroxylase activity and coumarin 7-hydroxylase (COH) activity, hepatocytes were incubated with 500 μM PNP or 100 μM coumarin according to the procedures previously described (Terner et al, in press). Assays were validated to be linear with time and proportional to number of cells. Production of metabolites was determined in duplicate by interpolation of standard curves made from authentic standards p-nitrocatechol and 7-hydroxycoumarin.

### 3MI Metabolism Assay

To measure 3MI metabolism in primary cultured hepatocytes, cells were incubated with 500 μM 3MI for 6 hours, after which an equal volume of acetonitrile was added to stop the reaction. The production of metabolites was then determined by HPLC analysis as previously described (Terner et al., in press). Formation of metabolites was determined by interpolation of standard curves made from authentic standards HMOI and 3MOI run in duplicate. The production of metabolites was found to be linear with time and number of cells.

### Statistical Analysis

The model used for the treatment effect of treatment on enzyme activity and 3MI metabolite production was as follows:

Yᵢⱼₖₗ=τᵢ×π_{j(i k)}×βₖ×β×τ₁×εᵢⱼₖₗ

Where Yᵢⱼₖₗ=chromatogram peak, τᵢ=inducer treatment, π_{j(i k)}=plate nested within treatment and within liver, βₖ=liver, βxτ₁=liver×treatment interaction and εᵢⱼₖₗ=the residual error of the experiments. Significant differences in PNP conversion, COH activity and 3MI metabolism were determined using the Dunnett's test for pairwise comparison using analysis of covariance (ANCOVA) in the Proc mixed program of the Statistical Analysis Software v8.0 (SAS institute, Carry, N.C.). Treatment related differences were determined with treatments as fixed effects and liver (treated as a block), liver×treatment interaction and plate within treatment within liver as random effects. The Satherthwaite approximation was used to calculate the denominator for the degrees of freedom given that variances were pooled for analysis. Activity and concentration of 3MI metabolite data was either logₑ or square root transformed to meet ANOVA assumptions of residual normality and homogeneous variance. The resulting analysis was examined using the Shapiro-Wilkes' test (Proc univariate normal) and by graphical interpretation of the residual plots. For all comparisons a type I error rate of α=0.05 was used unless otherwise stated. All results are presented as averages±standard errors, while all graphs are presented using the normalized raw data for easier illustration.

### Results and Discussion

### Effect of Inducers (Hormones/CAR/PXR Agonists) on CYP2E1 and CYP2A6 Activities and on 3MI Metabolism in 3-Week old Male and Adult Boar Hepatocytes

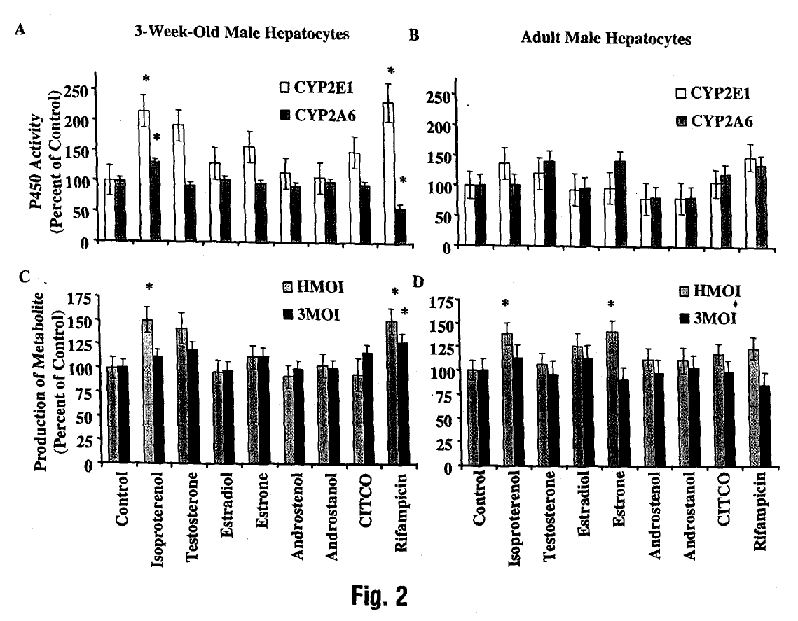

Previous research has shown an effect of castration on the accumulation of 3MI in fat of adult male pigs. Therefore, it is likely that a testis-derived factor is involved in altering liver metabolizing enzymes in the livers of males. Therefore, we wanted to determine the effects of various hormones on the expression of CYP2E1 and CYP2A6 and on 3MI metabolism in 3-week old male and adult male pig hepatocytes. As shown in FIG. 2, treatment of 3-week old male pig hepatocytes for 24 hours with isoproterenol resulted in a 215±26% (P<0.05) and 131±6% (P<0.05) increase in CYP2E1 and CYP2A6 activities respectively. An increase of 150±13% (P<0.05) in the production of the 3MI metabolite HMOI was also observed; however, the production of 3MOI was unaffected. In adult male hepatocytes, isoproterenol treatment did not alter CYP2E1 or CYP2A6 activity levels or 3MOI production, but did result in a 139±12% increase in HMOI production.

Treatment of 3-week old male pig or adult male hepatocytes with testosterone, estradiol, estrone, androstenol or androstanol did not alter CYP2E1 or CYP2A6 activity. Similar results were obtained for DHEA and estrone sulphate (data not shown). These hormone treatments did not affect the metabolism of 3MI as indicated by the production of HMOI or 3MOI in 3-week old male hepatocytes. However, in adult male hepatocytes treatment for 24 hours with estrone did result in a 142±12% (P<0.01) fold increase in HMOI production while not affecting 3MOI production.

Furthermore we wanted to determine the effects of nuclear receptor inducers for CAR (CITCO) and PXR (rifampicin) on the expression of these enzymes and on the metabolism of 3MI. We found that CITCO at 1.0 μM did not increase CYP2E1 or CYP2A6 activity, or affect 3MI metabolism in 3-week old male or adult male hepatocytes. In contrast, rifampicin a reported PXR agonist did result in a 233±32% (P<0.05) increase in CYP2E1 activity and a 43±7% (P<0.01) decrease in CYP2A6 activity in 3-week old male hepatocytes, with a concurrent increase of 151±14% (P<0.05) and 128±10% (P<0.05) in HMOI and 3MOI production. In adult male hepatocytes no significant affects of rifampicin were observed.

In 3-week old male hepatocytes we have previously demonstrated that CYP2E1 is of greater importance than CYP2A6 in the metabolism of 3MI (Terner et al. in press). Here we found that treatment of hepatocytes from 3-week old males with both isoproterenol and rifampicin increased the metabolism of 3MI as indicated by the production of the HMOI and 3MOI metabolites. However, these effects were attenuated in adult male hepatocytes. Also, while we did not observe an alteration of CYP2E1 and CYP2A6 activities by estrone treatment, we did demonstrate an increase in the production of HMOI, which suggests that other enzymes influenced by estrone treatment may be involved in the metabolism of 3MI.

Three-week old male pigs were used for the isolation of hepatocytes as hormone levels peak at this stage of development and are within the range of adult male boars, before dropping-off until puberty is reached (Schwarzenberger et al., 1993). Herein, we found that treatment of 3-week old male hepatocytes with isoproterenol resulted in an increase in both CYP2E1 and CYP2A6 activity levels while increasing HMOI production. In adult male hepatocytes an independent increase in HMOI production mediated by either CYP2E1 or CYP2A6 was observed. Isoproterenol has been shown to stimulate the production of cellular cAMP levels as well as to increase various other P450s in mice (Viitala et al., 20001). Increases in cAMP levels are also associated with an increment in steroidogenic P450s (Waterman and Bishchof, 1996) and an additive effect of cAMP stimulation and the treatment with the classical P450 inducer phenobarbital has been observed in primary hepatocytes (Salonpää et al., 1994). Together these observations suggest that the increase in HMOI metabolism may be due to a general increase in total P450s within the system and may not be specific to CYP2E1 or CYP2A6 activities.

Treatment of 3-week old male hepatocytes with rifampicin, a pig PXR receptor agonist (Moore et al., 2002), did lead to an increase in CYP2E1 activity and a decrease in CYP2A6 activity, while concurrently increasing the production of the 3MI metabolites HMOI and 3MOI. This demonstrates that these P450s are differentially regulated by PXR and supports our previous observations suggesting that an alteration of CYP2E1 activity is of greater significance than CYP2A6 in the metabolism of 3MI in 3-week old male hepatocytes (Terner et al., in press). However, rifampicin treatment of adult male hepatocytes did not influence CYP2E1 and CYP2A6 activities or 3MI metabolism. This suggests that either the PXR receptor does not influence the metabolism of 3MI in adult hepatocytes, or adult male hepatocytes are unresponsive to the effects of rifampicin or the levels of PXR are insufficient to respond to the inducers.

The increase in HMOI production by estrone treatment independently of CYP2E1 and CYP2A6 activities in adult male hepatocytes is a key observation. In adult male pigs fat levels of estrone are associated with the simultaneous increase in 3MI (Zamaratskaia et al., 2005). Estrone is known to mediate gene expression via the estrogen receptor (ER) (Archer et al., 1986). In addition, estrone has been shown to modulate the expression of other nuclear receptors, such as CAR, by increasing the NR1 enhancer element of murine CAR (Kawamoto et al., 2000). Conversely, mouse CAR activation has also been shown to inhibit ER signaling mechanisms (Min et al., 2002). Together these observations suggest that there is a network of cross-talking nuclear-receptors that regulate gene expression, which need to be further characterized in pigs. Furthermore, while we observed an effect directly on hepatocytes it also possible that hormones could influence other organs, which in turn affect liver gene expression. For instance, Liddle et al. (1998) investigated the effects of hormones on CYP3A4 regulation in cultured human hepatocytes and found that hormones were the major determinant of constitutive expression. They also suggest that sex hormones may not affect hepatocytes directly, but instead influence P450 expression in the liver by influence the release of growth hormone from the pituitary, that consequently affect P450 expression.

### REFERENCES

Archer T K, Tam S P, and R G Deeley (1986) Kinetics of estrogen-dependent modulation of apolipoprotein A-I synthesis in human hepatoma cells. J Biol Chem 261:5067-74.
Babol J, Squires E J, and E A Gullett (1996) Investigation of factors responsible for the development of boar taint. Food Research International 28:573-581.
Babol J, Squires E J and K Lundstrom (1998) Hepatic metabolism of skatole in pigs by cytochrome P4502E1. J Anim Sci 76:822-828.
Babol J, Squires E J and K Lundstrom (1999) Relationship between metabolism of androstenone and skatole in intact male pigs. J Anim Sci 77: 84-92.
Bonneau M (1997) Boar taint in entire male pigs—a review. Pig News and Information 18:15-18.
Chen Y, Ferguson S S, Negishi M, and J A Goldstein (2004) Induction of human CYP2C9 by rifampicin, hyperforin, and phenobarbital is mediated by the pregnane x receptor. J Pharmacol Exp Ther 308: 495-501.
Claus R, and B Hoffman (1980) Oestrogens, compared to other steroids of testicular origin, in blood plasma of boars. Acta Endocrinologica 94: 404-411.
Claus R, Weiler U, and A Herzog (1994) Physiological aspects of androstenone and skatole formation in the boar—a review with experimental data. Meat Science 38:289-305.
Desai P B, Nallani S C, Sane R S, Moore L B, Goodwin B J, Buckley D J and A R Buckley (2002) Induction of cytrochrome P450 3A4 in primary human hepatocytes and activation of the human pregnane x receptor by tamoxifen and 4-hydroxytamoxifen. Drug Metab Dispos 30: 608-612.
Diaz G J and E J Squires (2000a) Metabolism of 3-methylindole by porcine liver microsomes: Responsible cytochrome P450 enzymes. Toxicol Sci 55:284-292.
Donato M T, Castell J V and M J Gómez-Lechón (1998a) The coumarin 7-hydroxylation microassay in living hepatic cells in culture. Alternatives to Laboratory Animals 26:213-223.
Donato M T, Gómez-Lechón M J, Jover R, Nakamura T and J V Castell (1998b) Human hepatocyte growth factor down-regulates the expression of cytochrome P450 isozymes in human hepatocytes in primary culture. J Pharmacol Exp Ther 284:760-767.
Doran E, Whittington F W, Wood J D, and J D McGivan (2002) Cytochrome P450IIe1 (CYP2E1) is induced by skatole and this induction is blocked by androstenone in isolated pig hepatocytes. Chem-Biol Interact 140:81-92.
Forman B M, Tzameli I, Choi H S, Chen J, Simha D, Seol W, Evans R M and D D Moore. (1998) Androstane metabolites bind to and deactivate the nuclear receptor CAR-b. Nature 395: 612-615.
Friis C (1992) Distribution, metabolic fate and elimination of skatole in the pig. In: Measurement and Prevention of Boar Taint in Entire Male Pigs, In: Proc. EAAP Working Group: Production and Utilization of Meat from Entire Male Pigs, Roskilde, Denmark.
Friis C (1995) Is boar-taint related to sex differences or polymorphism of skatole metabolism, In: Proc. EAAP Working Group: Production and Utilization of Meat from Entire Male Pigs, United Kingdom.
Kawamoto T, Kakizaki S, Yoshinari K, and M Nakajima (2000) Estrogen activation of the nuclear orphan receptor CAR (constitutive active receptor) in induction of the mouse CYP2b10 gene. Mol Endocrinol 14: 1897-1905.
Liddle C, Goodwin B J, George J, Tapner M and G C Farrell (1998) Separate and interactive regulation of cytochrome P450 3A4 by triiodothyronine, dexamethasone, and growth hormone in cultured hepatocytes. Clin Endo Metabol 83: 2411-2416.
Maglich J M, Parks D J, Moore L B, Collins J L, Goodwin B, Billin A N, Stoltz C A, Kliewer S A, Lambert M H, Willson T M and J T Moore (2003) Identification of a Novel Human Constitutive Androstane Receptor (CAR) Agonist and Its Use in the Identification of CAR Target Genes. J Biol Chem 278:17277-83.
Maglich J M, Stoltz C M, Goodwin B, Hawkins-Brown D, Moore J T, and S A Kliewer (2002) Nuclear pregnane x receptor and constitutive androstane receptor regulate overlapping but distinct sets of genes involved in xenobiotic detoxification. Mol Pharmacol 62:638-46.
Min G, Kim H, Bae Y, Petz L, and J K Kemper (2002) Inhibitory cross-talk between estrogen receptor (ER) and constitutively activated androstane receptor (CAR). CAR inhibits ER-mediated signaling pathway by squelching p160 coactivators. J Biol Chem 277(37):34626-33.
Moore L B, Maglich J M, McKee D D, Wisely B, Willson T M, Kliewer S A, Lambert M H and J T Moore (2002) Pregnane X Receptor (PXR), Constitutive Androstane Receptor (CAR), and Benzoate X Receptor (BXR) Define Three Pharmacologically Distinct Classes of Nuclear Receptors. Mol Endocrinol 16 977-86.
Moore J T, Moore L B, Maglich J M, and S A Kliewer (2003) Functional and structural comparison of PXR and CAR. Biochim Biophys Acta 1619:235-8.
Moran F M, Ford J J, Corbin C J, Mapes S M, Njar V C, Brodie A M and A J Conley (2002) Regulation of microsomal P450, redox partner proteins, and steroidogenesis in the developing testes of the neonatal pig. Endocrinology 143: 3361-3369.
Reinach B. de Sousa G, Dostert P, Ings R, Gugenheim J and R Rahmani (1999) Comparative effects of rifabutin and rifampicin on cytrochromes P450 and UDP-glucuronosyl-transferases expression in fresh and cryopreserved human hepatocytes. Chemico-Biological Interac 121: 37-48.
SalonpääP, Pelkonen O, Kojo A, Pasanen M, Negishi M, and H Raunio (1994) Cytochrome P4502A5 expression and inducibility by phenobarbital is modulated by cAMP in mouse primary hepatocytes. Biochem Biophys Res Commun 205:631-7.
Schwarzenberger F, Toole G S, Christie H L and J I Raeside (1993) Plasma levels of several androgens and estrogens from birth to puberty in male domestic pigs. Acta Endocrinol 128:173-177.
Sinclair P A, Hancock S, Gilmore W J, and E J Squires (2005) Metabolism of the 16-androstene steroids in primary cultured porcine hepatocytes. J Steroid Biochem Mol Biol 96:79-87.
Squires E J and M Bonneau (2004) Boar taint, in Control Encyclopedia of Meat Sciences (Jensen W K, Devine C and M Dikeman eds). Article 9, Academic Press, Oxford.
Squires E J and K Lundstrom (1997) Relationship between cytochrome P4502E1 in liver and levels of skatole and its metabolites in intact male pigs. J Anim Sci 75:2506-2511.
Terner M A, Gilmore W J, Lou Y and E J Squires (XXXX) The role of CYP2A6 and CYP2E1 in the metabolism of 3-methylindole in primary cultured porcine hepatocytes. Drug Metab Dispos {accepted with revisions}
Viitala, P, K Posti, A Lindfors, O Pelkonen, and H Raunio (2001) cAMP mediated upregulation of CYP2A5 in mouse hepatocytes. Biochem Biophys Res Commun 80: 761-767.
Waterman M R and L J Bischof (1996) Mechanisms of ACTH(cAMP)-dependent transcription of adrenal steroid hydroxylases. Endocr Res 22:615-20.
Yost G S (1989) Mechanisms of 3-methylindole pneumotoxicity. Chem Res Toxicol 2:273-279.
Zamaratskaia G, Babol J, Madej A, Squires E J and K Lundstrom (2004) Age-related variation of plasma concentrations of skatole, androstenone, testosterone, oestradiol-17 beta, oestrone sulphate, dehydroepiandrosterone sulphate, triiodothyronine and IGF-1 in six entire male pigs. Reprod Domest Anim 39:168-72.
Zamaratskaia G, Madej A, Babol J, Squires E and K Lundstrom (2005) Free oestrone in adipose tissue and its relation to androstenone and skatole in entire male pigs. Reprod Domest Anim 40:156-60.

### EXAMPLE 3

### The Role of Nuclear Receptor CAR in Boar Taint

### Abstract

The boar taint phenotype is caused by an accumulation of androstenone and/or skatole in pig fat causing an off or fecal odor in the meat. The nuclear receptor CAR (Constitutive Active/Androstane Receptor) controls the metabolism of lipophilic compounds like hormones and drugs by regulating the expression of phase I and phase II metabolic enzymes. CAR may also be involved in the regulation of the metabolism of boar taint compounds. The goal of this project was to characterize pig CAR (pgCAR) and determine if it was regulated by the same hormonal ligands as in human and mouse; further to determine if this regulation is involved in the accumulation of androstenone and/or skatole in pig fatty tissues. We have shown that pig CAR is 87% homologous to human CAR (hCAR), and it could be used as a better model species than mouse for CAR studies in humans, since pgCAR responds to the same ligand treatments and is more sensitive than hCAR. Pig CAR has several novel hormonal ligands that cause significant repressions of gene expression in the luciferase reporter assay; these ligands include hormones in the Δ16 pathway: 5α-androsten-3β-ol, 5,16-androstadien-3β-ol, and the potent androgens 5α dihydrotestosterone (5α-DHT) and 5β-DHT. These compounds may repress the expression of genes involved in the metabolism of boar taint compounds. There are several alternative spliced form of pgCAR, and only the wild type form is capable of nuclear translocation and gene activation. Using boar hepatocytes as a model, we found that the known human CAR activators CITCO and phenytoin induce CAR activity and reporter gene expression. Further, genes involved in androstenone, androgen, and skatole metabolism are regulated by CAR in pig hepatocytes; these genes include 3α-hydroxysteroid dehydrogenase (3α-HSD), 3β-HSD, SULT2A1, UDP glucuronyltransferase 2B (UGT2B), CYP2A6 and CYP2E1. In a comparison of Low vs. High androstenone boars, the expression of 3β-HSD was significantly higher in low androstenone boars. These finding indicate that genes encoding enzymes responsible for the metabolism of boar taint compounds are regulated by pgCAR, and 5α-DHT and 5α-androsten-3β-ol are capable of repressing the ability of CAR to activate gene expression. Therefore, CAR likely plays a role in hormonal homeostasis and may be linked to the accumulation of boar taint compounds in boars.

### Introduction

The nuclear receptor CAR (Constitutive Active/Androstane Receptor, NR113) regulates the transcription of genes responsible for the metabolism of steroids, drugs and xenobiotic compounds (Handschin and Meyer 2003). CAR binds as a dimer with RXR to specific hexametric DNA sequences in the 5′-regulatory region of phase I and phase II enzymes (Auerbach, Ramsden et al. 2003). The CYP450 enzymes metabolise a wide variety of lipophilic compounds allowing for their activation or excretion. Nuclear receptor CAR binding sites have been identified in several CYP450 genes, including CYP3A4 and CYP2B6 that metabolize lipophilic drugs, hormones, bile, and cholesterol. (Handschin and Meyer 2003) The CYP450 promoter regions may have several response element (RE) sites allowing for activation by different nuclear receptors. CAR has been shown to possess overlapping RE binding affinity with its closest relative PXR (Pregnane X receptor); additional overlap exists with FXR (famesoid X receptor) (Kast, Goodwin et al. 2002) and several other nuclear receptor superfamily members (Handschin and Meyer 2003).

Although CAR competes with other nuclear receptors for DNA response element binding sites, it has evolved a very different approach to gene transcriptional activation. Constitutive androstane receptor was previously named constitutive active receptor for its high basal activity in the absence of any bound ligand. Researchers were also surprised to discover that stereospecific forms of the mammalian pheromone androstenol were able to completely inhibit mouse CAR RE binding activity (Forman, Tzameli et al. 1998). Androstanol is thus considered an inverse agonist of CAR due to its negative effect on regulation (Picard 1998). Androstenone is converted to 3α-androstenol by the 3α-HSD enzyme (Dufort, Soucy et al. 2001). CAR has evolved a specific inverse agonist ligand binding domain (LBD) allowing very few compounds to disrupt its constitutive transcriptional activation. In mouse, 5α-androst-16-an-3α-ol and 5α-androst-16-en-3α-ol (5α-androst-16-an/en-3α-ol) are capable of preventing CYP450 expression by decreasing the interaction of CAR with coactivators, such as GRIP-1 or SRC-1 (Forman, Tzameli et al. 1998) (Min, Kemper et al. 2002). 5β-androst-16(an/en)-3α-ol, which is identical to 5α-androst-16(an/en)-3α-ol except the orientation of the hydrogen at carbon 5, des not repress CAR RE binding (Forman, Tzameli et al. 1998). CYP3A4 is the most abundantly expressed CYP450 in human liver; females have been shown to have significantly higher levels of CYP3A4 expression than males (Wolbold, Klein et al. 2003). Sexual dimorphism of CYP3A4 expression could be explained by the regulation of CAR by inverse hormone agonists.

Studies comparing human CAR (hCAR) to mouse CAR (mCAR) have shown major differences between the receptors in the two species. Mouse CAR is translocated to the nucleus when phenobarbital or the synthetic TCPOBOP treatment is used; these compounds do not cause the translocation of hCAR (Moore, Maglich et al. 2002). Few compounds, including 6-(4-chlorophenyl)imidazo[2,1-b][1,3]thiazole-5-carbaldehyde O-(3,4-dichlorobenzyl)oxime (CITCO) have been shown induce nuclear translocation of hCAR, like phenobarbital does for mCAR (Maglich, Parks et al. 2003).

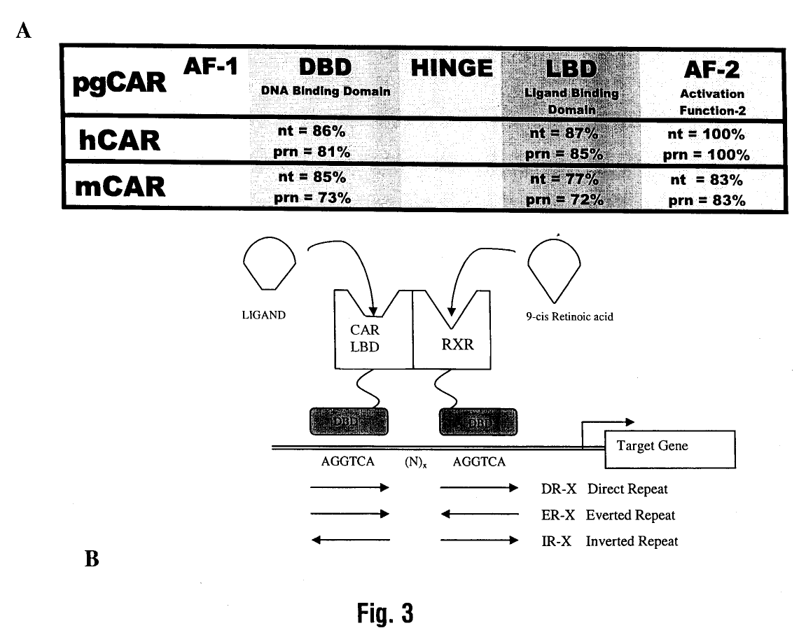

CAR binds DNA as a heterodimer with the retinoid-X receptor (RXR) (Baes, Glulick et al. 1994). Mutagenesis of RXR inhibits dimerization of certain nuclear receptors; however CAR dimerization was not affected in this study (Lee, Lee et al. 2000) CAR has been shown to bind to direct repeat DNA response elements DR-3, DR-4 and DR-5. Binding specificity of other nuclear receptors depends on the number of base pairs (bp) between the repeated DNA sequences. For example DR-4 has four bp of any nucleotide (N) between the direct repeat sequences (AGGTCANNNNAGGTCA). Other REs recognized by CAR are everted repeat-6 (ER-6) and inverted repeat-8 (IR-8) sequences seen in FIG. 3.

Researchers have recently identified alternative splicing isoforms in both human and mouse CAR (Auerbach, Ramsden et al. 2003), (Choi, Chung et al. 1997), (Jinno, Tanaka-Kagawa et al. 2004). In-frame isoforms have been identified for hCAR; these isoforms have impaired or no transcriptional activation ability. Isoforms for mCAR and now pig CAR (pgCAR; our work here) have been identified that contain out of frame mutations resulting in the loss of the AF-2 domain required for nuclear translocation. The function of CAR alternative splicing is still not well understood.

The nuclear receptor CAR from pig has been cloned and expressed by our lab and may be a factor in the development of boar taint. Boar taint is a complex trait affecting primarily intact male pigs; the phenotype is characterized by the accumulation of pungent smelling androstenone and/or fecal smelling skatole in the fat causing a strong off odor of the meat upon cooking. Boar taint is mostly prevented by castration of the male pigs shortly after birth; this eliminates the source of androstenone and has also been shown to reduce skatole levels. Cytochrome P450 2E1 and 2A6 are the major metabolizing enzymes for skatole; their regulation is still under investigation. The phase II sulfotransferase enzymes convert androstenone and skatole to water soluble metabolites preventing their deposition in the fat. In human and mouse there is evidence that CAR and PXR regulate these classes of enzymes. These observations suggest the boar taint phenotype may be in part regulated by pgCAR.

### Objective 1

### Cloning and Sequencing of pig CAR

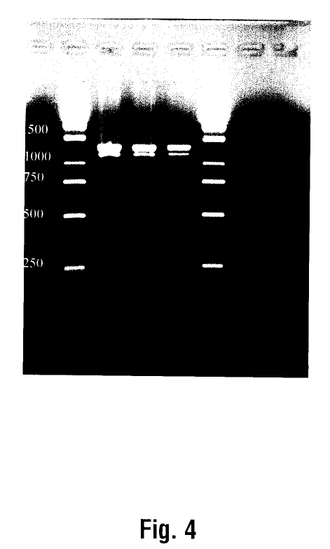

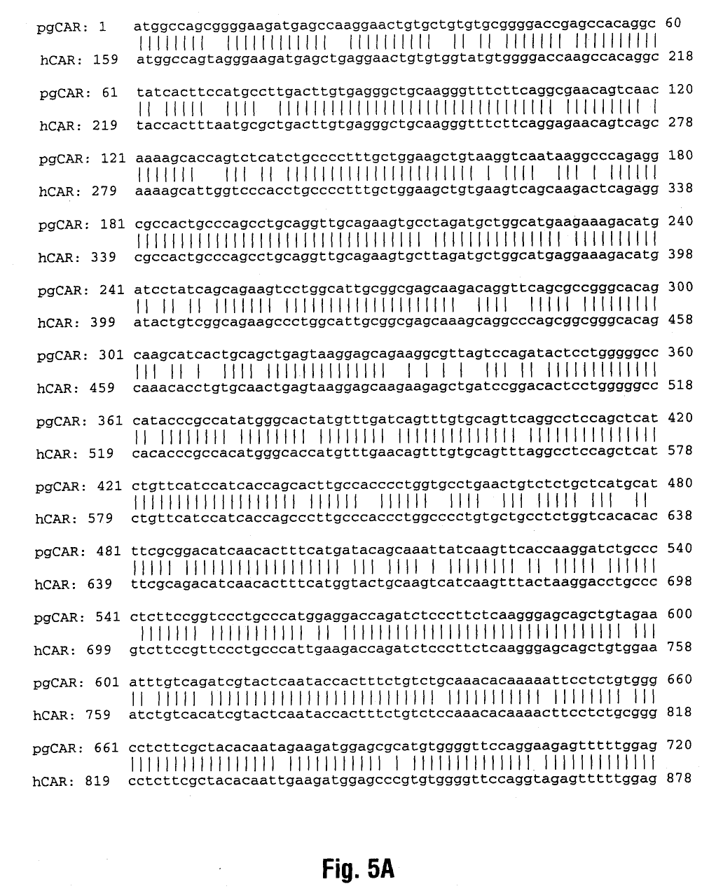

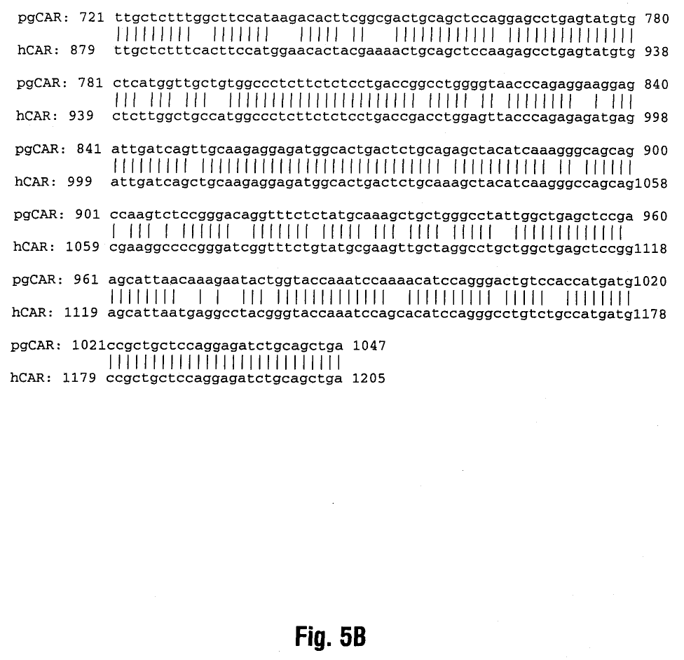

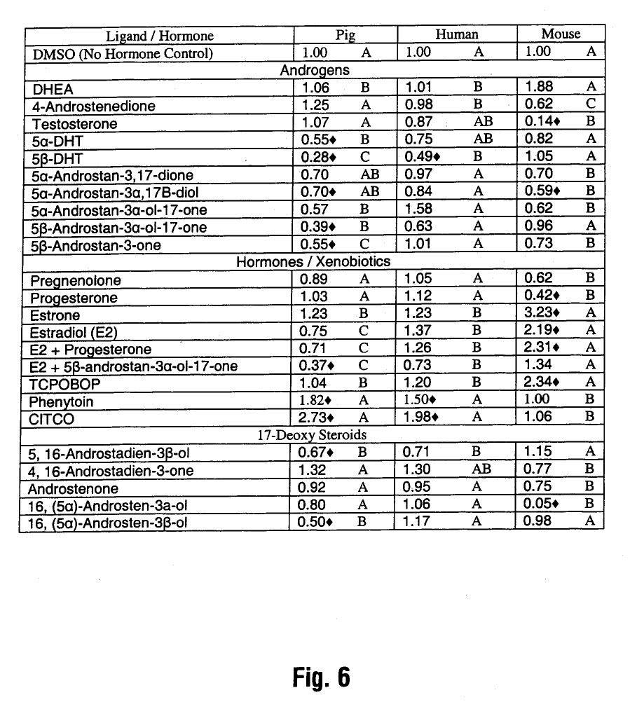

Pig CAR was cloned using a computational genomics approach. Human CAR gi:32189358 was BLAST searched against the Sus. Scrofa EST database; two sequences with high sequence homology were identified and contained partial pgCAR sequences from the 5′ untranslated region (UTR) (NCBI gi:10874174) and 3′UTR (NCBI gi:37791680). Forward and reverse primers were designed to amplify the full coding sequence of pgCAR. FIG. 4 shows the PCR fragments generated by the initial pgCAR primers. The resulting PCR product was cloned into the pCR 3.1 cloning plasmid for sequencing. FIG. 5 shows a nucleotide alignment BLAST search, which shows human CAR NR113 as having the closest homology (86%) at the nucleotide level, and 75% at the protein level. The complete breakdown of percent homology between different domains of pgCAR and hCAR or mCAR is shown in FIG. 1A. The pig and human CAR are identical in length at the nucleotide and amino acid levels, while the mouse utilizes an upstream ATG which adds length to the AF-1 region. The ligand binding domain of human is 85% similar to pig at the amino acid level, while the ligand binding domain of pgCAR is 72% homologous to mCAR. The CAR activation assays (FIG. 6) shows pig and human CAR respond to the same ligands, likely due to the close homology of their ligand binding domains. Further, the Activation Function 2 (AF2) domain that is required for nuclear translocation is 100% identical between human and pig CAR. Studies involving the ligand CITCO hypothesize that the interaction of the AF-2 domain with CITCO results in nuclear translocation.

### Objective 2

### Identifying Alternatively Spliced Isoforms of CAR

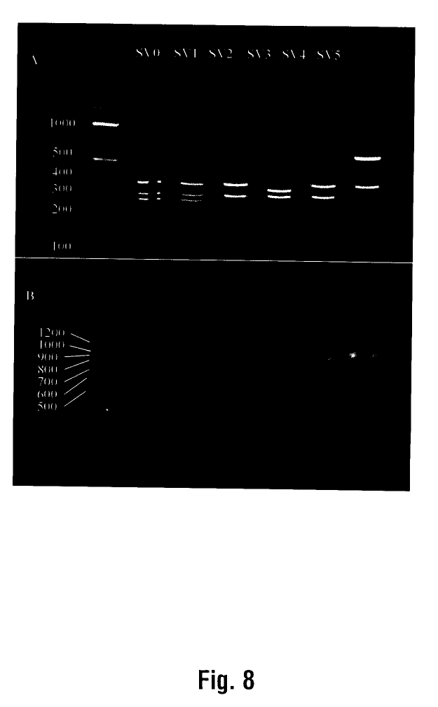

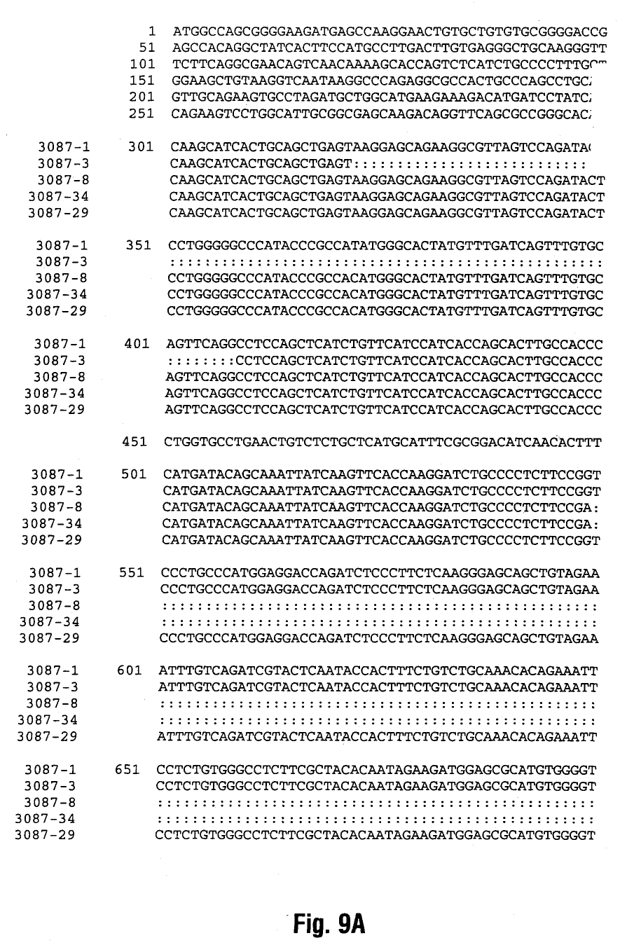

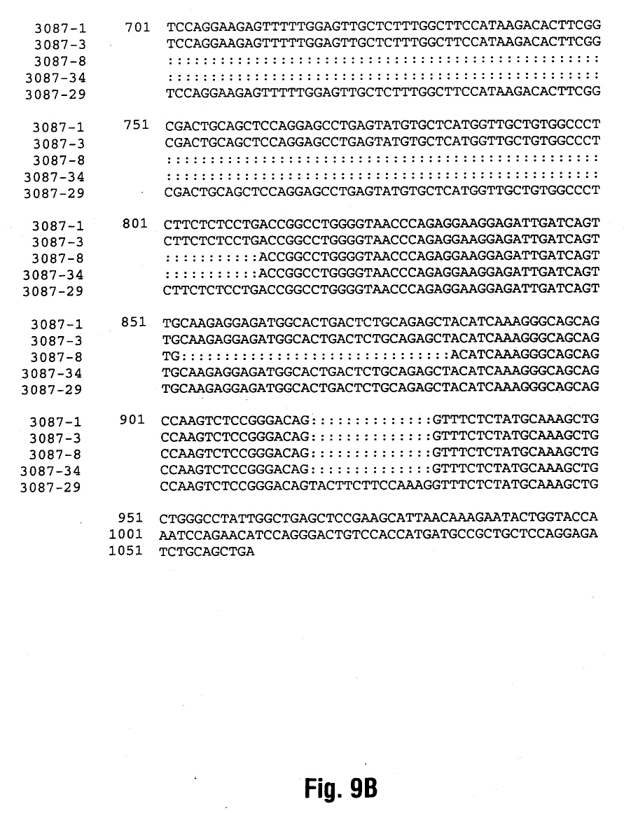

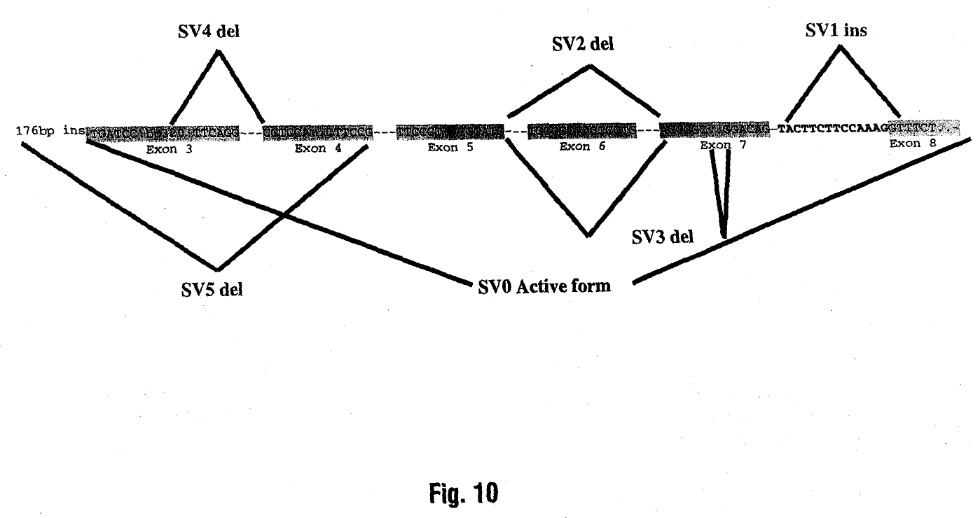

Recent publications have identified alternatively spliced isoforms of both human and mouse CAR, and alternative splicing appears to be an important means of regulating CAR at the transcript level (Jinno, Tanaka-Kagawa et al. 2004). Alternative splicing dramatically decreases CAR's ability to bind DNA and activate transcription (Auerbach, Ramsden et al. 2003; Savkur, Wu et al. 2003). We therefore identified alternatively spliced isoforms of pgCAR from liver cDNA. Liver RNA is first DNase I treated to remove genomic DNA and then reverse transcribed to cDNA. The cDNA was then amplified using pgCAR expression plasmid primers and a proofreading DNA polymerase and then cloned into the pCR3.1 plasmid and transfected into chemically competent E. coli. Bacterial colonies are randomly picked from the ampicillin selection LB agar plates and added to a colony PCR mix to amplify the pgCAR insert in the plasmids. The resulting amplicon was double digested with NcoI and NciI to produce five fragments of 353, 292, 265 and 148 bp for the wild type CAR transcript. The digested DNA is separated on a 2% agarose gel and alternatively spliced isoforms produce bands that differ from the wild type banding pattern. FIG. 8 shows six alternative spliced isoforms of pgCAR. Each unique clone was DNA sequenced and FIG. 9 compares nucleotide alignments of all pgCAR isoforms. Sequence translation (data not shown) shows all alternatively spliced isoforms have frameshift mutations. FIG. 10 compares exon junction points for the identified isoforms. The wild type CAR isoform SV0 is the predominant form of pgCAR and all identified alternative splicing forms of pgCAR resulted in frameshifts in the coding sequence. These frameshifts disrupt the AF-2 region; therefore none of the splice forms are capable of nuclear translocation. It has been hypothesized that alternative splicing of CAR is another regulatory level for CAR. For example, tissues such as the heart express CAR, but it is all is spliced to non-functional forms. Since in pig alternative splicing occurs through the entire coding region (FIGS. 9 & 10), quantifying the percent of active transcript or splice forms will be problematic.

### Objective 3

Comparative Analysis of pgCAR, hCAR and mCAR Regulation by Hormonal and Xenobiotic Ligands.

The dual luciferase reporter assay (Promega) was used as a screening tool to identify ligands (hormone and xenobiotic compounds) that regulate the nuclear receptor CAR. The dual luciferase assay consists of 3 plasmids. A pgCAR or hCAR or mCAR expression plasmid containing the full length wild type isoform is used together with a reporter construct, consisting of the CAR response gene promoter element from CYP2B6 regulating the expression of the firefly luciferase gene. The third plasmid is an internal control plasmid that is driven by a strong viral promoter upstream of the Renella luciferase gene; it is used to determine transfection efficiency and to normalize replicates. The Promega Dual-Luciferase Reporter Assay System (Cat#E11960) was used to determine the hormones and xenobiotics that modulate the CAR responsive genes by measuring the amount light produced by the firefly luciferase gene construct.

The dual luciferase assay method uses HepG2 cells (that express RXR) plated at 50% confluence onto 96 well cell culture plates. The cells are transiently transfected after 1 day with pgCAR expression plasmid, reporter plasmid (containing the CAR response element) and the internal control plasmid, which are pooled treatments to ensure all samples receive the same amount of plasmids. On day two, control or ligand supplemented media are added to cells (ligands from FIG. 4 at 10 ug/ml in DMSO) for a total of n=15 for each ligand. On day four, the media is removed and cells are washed with PBS to remove detached cells and remaining media. The cell monolayer is then treated with Passive lysis buffer and the cell lysate is then transferred to a test tube for analysis. Luminometer readings are made on a Berthold Detection systems Sirius single tube luminometer with dual injectors. The test tube is loaded into the measurement chamber and the LARII luciferase reagent is injected into the cell lysate and a 10 second firefly luciferase relative light unit (RLU) measurement is recorded. The Stop & Glo reagent is then injected into the same tube and the Renella luciferase RLU is measured. The ratio of reporter to control luminescence is used to normalize transfection efficiencies between plates; the normalized values are then statistically analyzed by t-tests to determine how efficiently CAR activates the firefly luciferase reporter gene in response to ligand treatment.

A comparison between pgCAR, hCAR and mCAR identified common hormonal ligands. The results of this extensive comparative assay are shown in FIG. 6. Of the 24 ligands tested, pig:mouse:human responded significantly to 10:8:3 ligands respectively. Pig CAR responded more strongly to each of the three ligands that activated hCAR. Human and mouse CAR were not affected by any common tested ligands, while pgCAR and mCAR had one ligand in common. The response of pgCAR and hCAR to ligands was not significantly different for 13/24 ligands, hCAR and mCAR responses to ligand were not significantly different for 5/24 ligands tested, and the response of pCAR and mCAR were not significantly different for 3/24 ligands. The results of this assay indicate that pgCAR responds to ligands in a more similar manner to hCAR than does mCAR. Furthermore, pgCAR is more responsive to ligands tested than hCAR. Pig is therefore better model species than mouse for drug testing in humans, because it responds to a greater degree to the same compounds that activate hCAR. It may be that other pig nuclear receptor respond to ligands more strongly than the corresponding human receptor. On the other hand, mouse and rat CAR and possibly other xenosensing receptors respond strongly to ligands that the human receptors do not respond to; further, mouse and rat CAR do not respond to compounds that human CAR responds to.

Looking more closely at the pig CAR receptor, pgCAR is repressed by several parent compounds and metabolites of androstenone; of particular interest are 5α-DHT and 5α-androsten-3β-ol. Testosterone is converted to 5α-DHT predominately in the prostate and is responsible for the development of male characteristics including bulbourethral gland length. In the pig, the concentrations of 5α-androsten-3β-ol are approximately 5 times higher than androstenone in circulating plasma, although this can vary dramatically among different animals. 5α-Androsten-3β-ol is the only Δ16 metabolite excreted in the urine at a rate of 250 ug/L as a glucuronide metabolite. Little is known about 5β-DHT in boars; however, it is likely not a major metabolite since estrogen has been shown to inhibit 5β-reductase in pig and boars are known to exceed females in estrogen production.

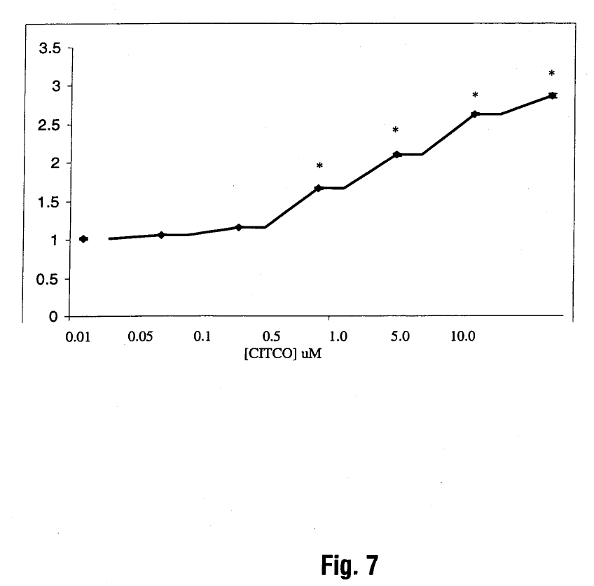

Since CITCO showed significant activation at the 10 uM level, a dose response experiment was conducted to determine at what concentration CITCO significantly activates the pgCAR receptor in HepG2 cells; this data is illustrated in FIG. 7. Significant activations were observed at starting at the 0.5 uM dose, Notably, this activation was observed over the constitutively high reporter gene levels seen after CAR transfection in HepG2 cells.

### Objective 4

Effects of Ligands on pgCAR in Primary Boar Hepatocytes using the Dual-Luciferase Reporter Assay

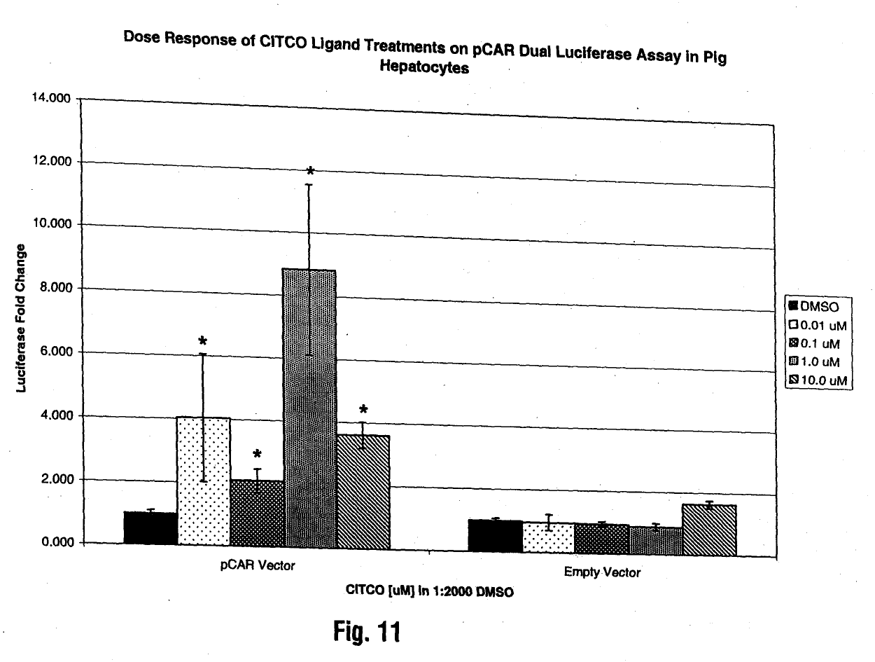

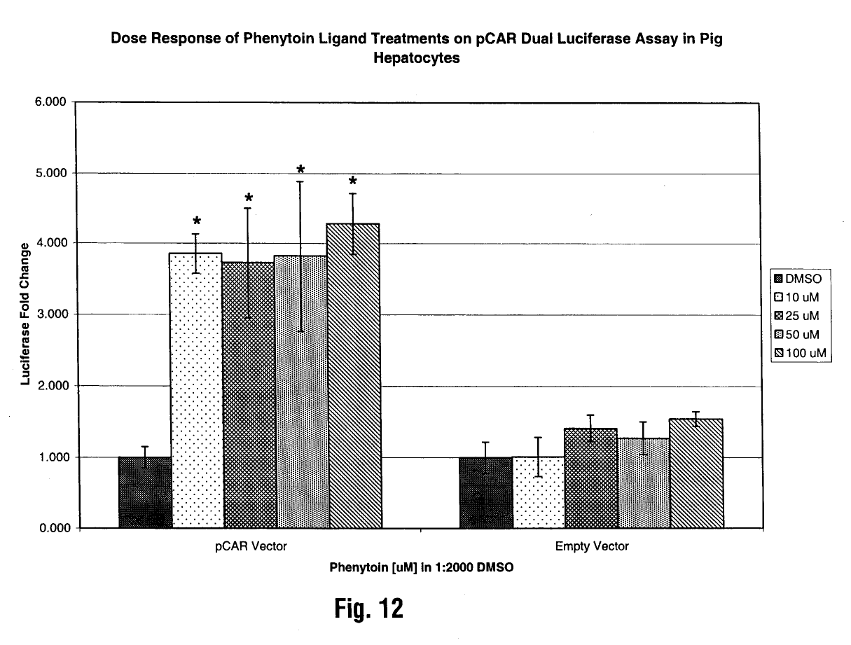

Transfection experiments using the HepG2 cell line identified compounds that were able to alter pgCAR gene regulation. Experiments performed on primary mouse and human hepatocytes indicate that gene regulation has additional levels of complexity, and CAR is not constitutively active in vivo. This experiment uses a similar methodology as objective 2 except primary boar hepatocytes were used as the cell line. Hepatocytes were transfected with a reporter construct containing the CAR responsive element, an internal control Renilla luciferase construct, and pgCAR or an empty vector as control. Control experiments without pgCAR were conducted to determine if endogenous pgCAR in the hepatocytes could activate the reporter construct. The results of these experiments show that the endogenous levels of pgCAR were unable to stimulate transcription of the luciferase reporter construct in the hepatocyte model system. However, when exogenous pgCAR expression plasmid is transfected, the pgCAR ligands CITCO and phenytoin activated the reporter construct as in HepG2 cells (FIGS. 11 and 12 respectively). Phenytoin is believed to activate CAR indirectly since it does not bind the ligand binding domain of CAR; this mechanism is the same as TCPOBOP in mouse. The cascade of events leading to gene activation remains unclear.

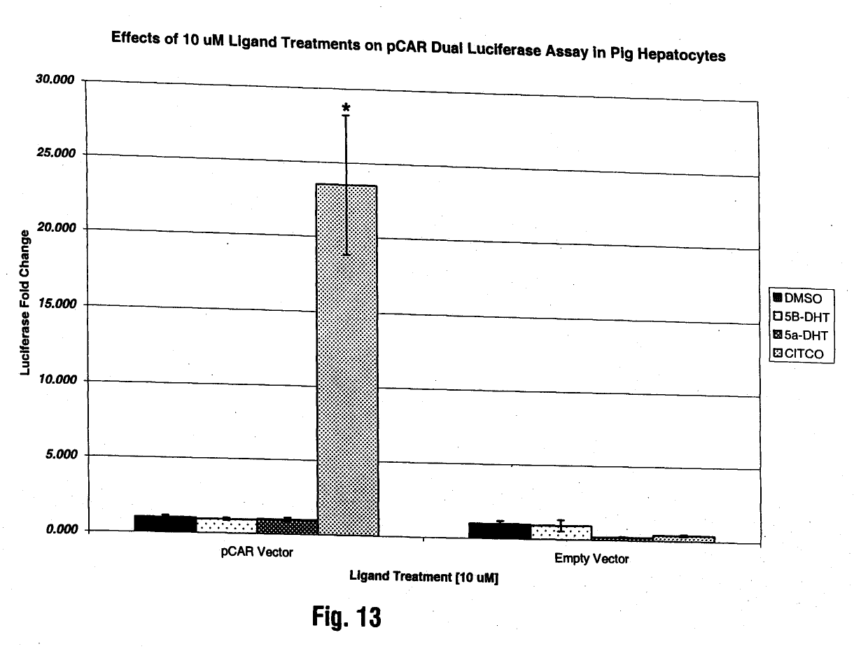

Compounds that repress reporter gene expression in the HepG2 cell model could not be detected in this experiment, since the no hormone DMSO treatments had extremely low basal levels, as seen in mouse and human hepatocyte experiments. Without high basal levels of luciferase expression in the no ligand controls, repressions were not detectable (FIG. 13).

### Objective 5

### Regulation of Phase I and Phase II Genes of Interest in the Metabolism of Androstenone and Skatole.

Primary boar hepatocytes were treated with seven different CAR ligands identified in objective 2 and 5, with DMSO used as the vehicle control. Primary cells were plated on 60 mm dishes and treated with ligand. The treatments were incubated for 8 or 10 hours and the RNA was extracted from cells in each treatment. Real Time PCR primers were designed for seven genes of interest CYP2A6, CYP2E1, 3β-HSD, 3α-HSD, SULT2A1, SULT1A1, UGT2B, CYP2B6 and pgCAR, with B-actin used as the housekeeping gene for ΔΔCT analysis. These genes were selected because they are known to be involved in androstenone and skatole metabolism, 3β-HSD and 3α-HSD are responsible for the phase I metabolism of androstenone to the more polar metabolites 5α-androsten-3α/β-ol. These metabolites are less lipophilic and have a C3 hydroxyl group that can be metabolized in Phase II by sulfotransferase SULT2A1 or glucuronidated by UGT2B. CYP2A6 and CYP2E 1 are involved in the Phase I metabolism of skatole and phenol sulfotransferase (SULT1A1) is responsible for the phase II metabolism by adding a sulfate group. CAR itself was screened to ensure the compounds did not regulate CAR expression. CYP2B6 is the model CAR upregulated gene; in CAR null mice CYP2B6 levels are virtually non-existent, so this gene was used as a CAR activation control.

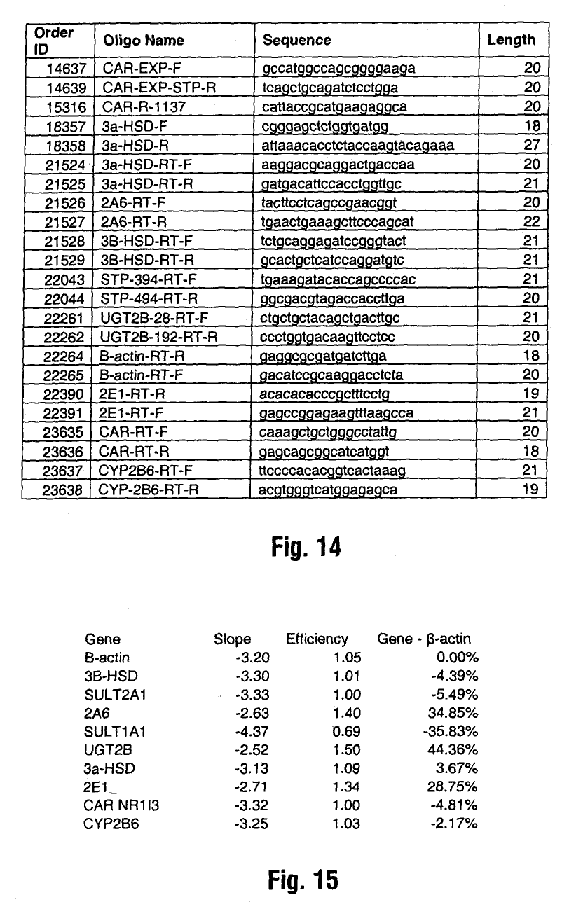

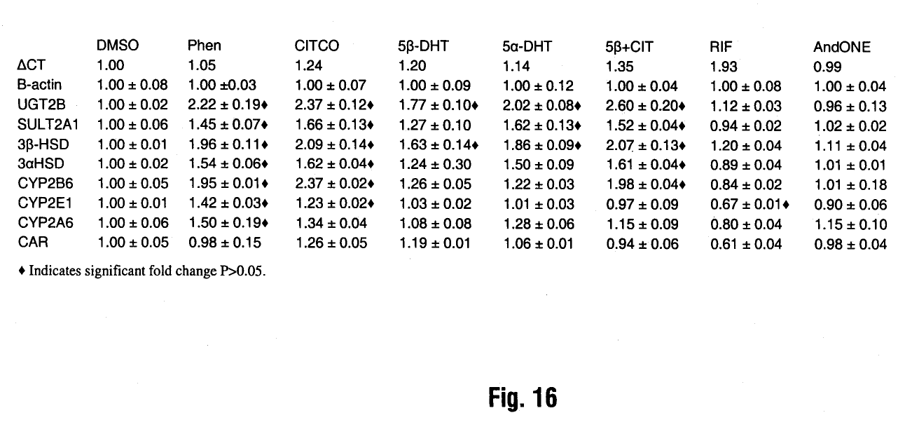

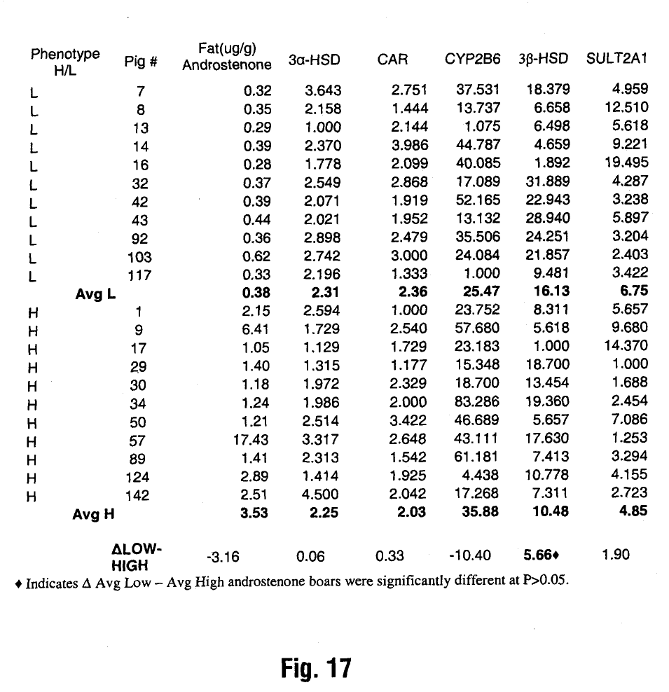

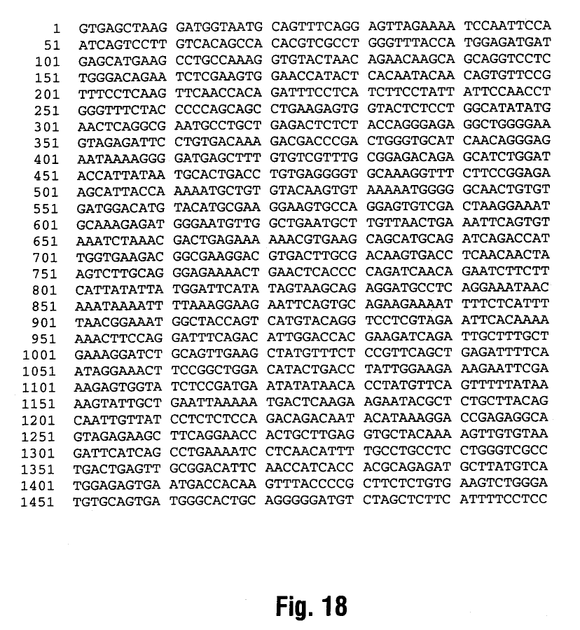

The designed primers and their efficiencies relative to B-actin are shown in FIGS. 14 and 15 respectively. The results of this experiment (FIG. 16) show that several ligand treatments modulate gene expression in a statistically significant manner. The results of this experiment indicate that CAR induces the expression of the CAR control gene CYP2B6 and this gene was not activated or repressed by the androgens 5α-DHT and 5β-DHT. All of the androstenone and skatole metabolizing enzymes were significantly activated when compared to the control, indicating that CAR is an important regulator of genes encoding these enzymes. The 5α-DHT and 5β-DHT treatments were included to determine if these compounds could repress gene activation as seen in the Dual luciferase reporter assay. UGT2B, SULT2A1 and 3β-HSD were all significantly activated by the 5α-DHT treatment. Since this is a potent androgen, it is hypothesized that these genes contain multiple nuclear receptor responsive elements, including the androgen receptor responsive element in their promoter regions. This is logical since androgen metabolism also requires these genes. In a comparison of B-actin expression between ligand treatments, only the rifampician (RIF) treatment had significantly higher B-actin expression compared to DMSO, indicating that only minor corrections were made in the analysis. This information is presented in FIG. 16 as ΔCT.

### Objective 6

Do High vs. Low Androstenone Boars Show Differential Gene Expression Patterns?

This final experiment was performed to determine if CAR regulated genes have lower expression levels in high androstenone boars than in low androstenone boars. Previously, SULT2A1 gene expression was shown to have a two-fold change in expression between high and low androstenone boars (high>1.0 in fat and low<0.5 in fat). The goal of this final experiment was to determine if other CAR regulated genes show similar expression patterns in the high and low androstenone boars. Eleven high and eleven low androstenone boars were selected for real time PCR analysis of SULT2A1, 3β-HSD, 3α-HSD, CAR, CYP2B6 and UGT2B using the ΔΔCT method of analysis. The results (FIG. 15) show only 3α-HSD expression is significantly higher in the low androstenone boars. SULT2A1 was 1.9 fold higher but this increase was not statistically significant.
Auerbach, S. S., R. Ramsden, et al. (2003). “Alternatively spliced isoforms of the human constitutive androstane receptor.” Nucl. Acids. Res. 31(12): 3194-3207.
Baes, M., T. Glulick, et al. (1994). “A New Orphan Member of the Nuclear Hormone Receptor Superfamily That Interacts with a Subset of Retinoic Acid Response Elements.” Molecular and Cellular Biology 14(3): 1544-1552.
Choi, H.-S., M. Chung, et al. (1997). “Differential Transactivation by Two Isoforms of the Orphan Nuclear Hormone Receptor CAR. ” J. Biol. Chem. 272(38): 23565-23571.
Diaz, G. and E. Squires (2000a). “The role of aldehyde oxidase in the hepatic metabolism of 3-methylindole in pigs.” J. Food Agric. Chem 48: 833-837.
Dufort, I., P. Soucy, et al. (2001). “Comparative biosynthetic pathway of androstenol and androgens.” The Journal of Steroid Biochemistry and Molecular Biology 77(4-5): 223-227.
Forman, B. M., I. Tzameli, et al. (1998). “Androstane metabolites bind to and deactivate the nuclear receptor CAR-b.” Nature 395: 612-615.
Handschin, C. and U. A. Meyer (2003). “Induction of Drug Metabolism: The Role of Nuclear Receptors.” Pharmacol Rev 55(4): 649-673.
Hansen, L., E. Larsen, et al. (1994). “Influence of stocking rate and faeces deposition in the pen at different temperatures on skatole concentration (boar taint) in subcutaneous fat.” Animal Production 59: 99-110.
Jinno, H., T. Tanaka-Kagawa, et al. (2004). “Identification of Novel Alternative Splice Variants of Human Constitutive Androstane Receptor and Characterization of Their Expression in the Liver.” Mol Pharmacol 65(3): 496-502.
Kast, H. R., B. Goodwin, et al. (2002). “Regulation of Multidrug Resistance-associated Protein 2 (ABCC2) by the Nuclear Receptors Pregnane X Receptor, Farnesoid X-activated Receptor, and Constitutive Androstane Receptor.” J. Biol. Chem. 277(4): 2908-2915.
Lee, S.-K., B. Lee, et al. (2000). “Mutations in Retinoid X Receptor That Impair Heterodimerization with Specific Nuclear Hormone Receptor.” J. Biol. Chem. 275(43): 33522-33526.
Maglich, J. M., D. J. Parks, et al. (2003). “Identification of a Novel Human Constitutive Androstane Receptor (CAR) Agonist and Its Use in the Identification of CAR Target Genes.” J. Biol. Chem. 278(19): 17277-17283.
Min, G., J. K. Kemper, et al. (2002). “Glucocorticoid Receptor-interacting Protein 1 Mediates Ligand-independent Nuclear Translocation and Activation of Constitutive Androstane Receptor in Vivo.” J. Biol. Chem. 277(29): 26356-26363.
Moore, L. B., J. M. Maglich, et al. (2002). “Pregnane X Receptor (PXR), Constitutive Androstane Receptor (CAR), and Benzoate X Receptor (BXR) Define Three Pharmacologically Distinct Classes of Nuclear Receptors.” Mol Endocrinol 16(5): 977-986.
Picard, D. (1998). “Two orphans find a home.” Nature.
Savkur, R. S., Y. Wu, et al. (2003). “Alternative splicing within the ligand binding domain of the human constitutive androstane receptor.”
Wolbold, R., K. Klein, et al. (2003). “Sex Is a Major Determinant of CYP3A4 Expression in Human Liver.” Hepatology 38(4): 978-988.

We claim:

1. A method for determining the susceptibility of a male pig to developing boar taint comprising:
(a) obtaining a sample from the pig; and
(b) detecting the levels of one or more nuclear receptors selected from the group consisting of (i) CAR, PXR, and/or FXR;
wherein high levels of CAR, PXR, and/or FXR indicates that the pig has a reduced susceptibility to developing boar taint.

2. A method according to claim 1 wherein the sample is selected from the group consisting of a liver or testis sample and blood.

3. A method according to claim 2 wherein levels of CAR, PXR, and/or FXR that are the same or higher than in a female control pig indicate that the pig has a reduced susceptibility to developing boar taint.

4. A method for reducing or preventing boar taint comprising: enhancing the metabolism of skatole or androstenone in a pig.

5. A method according to claim 4 wherein the activity of the CAR, PXR, and/or FXR is enhanced by administering
(a) a substance that increases the activity of the CAR, PXR, and/or FXR; or
(b) a substance that induces or increases the expression of the CAR, PXR, and/or FXR gene.

6. The method of claim 5 wherein said activity of CAR, PXR and/or FXR results in increased SULT2A1 activity.

7. The method of claim 6 wherein said method of enhancing the activity of CAR, PXR and/or FXR is by treatment with a ligand of CAR, PXR or FXR.

8. The method of claim 7 wherein said ligand is rifampicin and said nuclear receptor is PXR.

9. The method of claim 7 wherein said ligand is lithocholic acid and said nuclear receptor is FXR or PXR.

10. The method of claim 6 wherein said activity is enhanced by treatment with a inducer of CAR, PXR or FXR.

11. The method of claim 10 wherein said inducer is phenytoin and said nuclear receptor is CAR.

12. The method of claim 6 wherein said activity is enhanced by decreasing the concentration of the inhibitory ligands for CAR.

13. The method of claim 7 wherein said nuclear receptor is CAR and further wherein said ligand affects the activity of a protein involved in boar taint, said protein selected from the group consisting of: 3β-HSD, 3α-HSD, SULT2A1, UGT2B, CYP2A6, or CYP2E1

14. A method according to claim 6 wherein a nucleic acid sequence encoding a nuclear receptor CAR, PXR, and/or FXR is introduced into a pig.

15. A method of screening for a substance that enhances the activity of CAR, PXR, and/or FXR comprising assaying for a substance which selectively (i) enhances CAR, PXR, and/or FXR activity, or (ii) enhances transcription and/or translation of the gene encoding CAR, PXR, and/or FXR.

16. A method according to claim 15 comprising the steps of:
(a) reacting a ligand of CAR, PXR, and/or FXR and CAR, PXR, and/or FXR, in the presence of a test substance, under conditions such that CAR, PXR, and/or FXR is capable of binding the ligands to activate or inactivate the receptor and concomitantly increase the transcription of genes
(b) assaying for transcription of genes, unbound ligand, or unbound CAR, PXR, and/or FXR; and
(c) comparing to controls to determine if the test substance selectively enhances CAR, PXR, and/or FXR activity and thereby is capable of enhancing skatole or androstenone metabolism in a pig.

17. A method according to claim 16 comprising the steps of:
(a) culturing a host cell comprising a nucleic acid molecule containing a nucleic acid sequence encoding CAR, PXR, and/or FXR and the necessary elements for the transcription or translation of the nucleic acid sequence, and optionally a reporter gene, in the presence of a test substance; and
(b) comparing the level of expression of CAR, PXR, and/or FXR, or the expression of the protein encoded by the reporter gene with a control cell transfected with a nucleic acid molecule in the absence of the test substance.

18. A composition for reducing or preventing boar taint comprising administering an effective amount of a substance selected from the group consisting of:
(a) a substance that increases the activity of the CAR, PXR, and/or FXR receptor
(b) a substance that induces or increases the expression of the CAR, PXR, and/or FXR gene.

19. An isolated polynucleotide comprising a polynucleotide selected from:
(a) a polynucleotide encoding a CAR polypeptide of the sequences shown herein;
(b) a polynucleotide which encodes the polypeptide sequence shown herein,
(c) a polynucleotide which selectively hybridizes under stringent conditions to a polynucleotide of (a) or (b);
(d) a polynucleotide having at least 80% sequence identity with polynucleotides of (a) or (b); and
(e) a polynucleotide comprising at least 25 contiguous nucleotides from a polynucleotide of (a), (b), (c), or (d).

20. A polypeptide encoded by the polynucleotide of claim 19.

21. A method of identifying a polymorphism correlated with boar taint comprising the steps of:
obtaining a sample of genetic material from a pig, said sample comprising a nuclear receptor gene selected from the group consisting of: CAR, PXR, and FXR;
assaying for said nuclear receptor gene present in said sample for a polymorphism;
correlating whether a biologically significant association exists between said polymorphism and boar taint, androstenone metabolism, skatole metabolism or a compound involved therewith, in a pig of a particular breed, population or group whereby said pig can be characterized for said polymorphism.

22. A method of screening pigs to determine those more likely to have reduced boar taint comprising:
obtaining a sample of genetic material from said pig; and
assaying for the presence of a genotype in a nuclear receptor gene selected from the group consisting of CAR, FXR and PXR, in said pig, wherein said genotype is associated with favorable boar taint characteristics, said genotype characterized by the following:
a) a polymorphism in the CAR, FXR or PXR gene.
